SEQUENCE LISTING (32 sequences)

SEQ ID NO: 1 - protein, 13 aa, Artificial Sequence
  Description of Artificial Sequence  Synthetic Peptide
CAVSRDSGNTPLV

SEQ ID NO: 2 - DNA, 39 bp, Artificial Sequence
  Description of Artificial Sequence  Synthetic Primer
tgtgctgtgagcagggattcaggaaacacacctcttgtc

SEQ ID NO: 3 - protein, 13 aa, Artificial Sequence
  Description of Artificial Sequence  Synthetic Peptide
CASSPSGTGNIQY

SEQ ID NO: 4 - DNA, 39 bp, Artificial Sequence
  Description of Artificial Sequence  Synthetic Primer
tgtgccagcagccctagcgggacagggaacattcagtac

SEQ ID NO: 5 - protein, 11 aa, Artificial Sequence
  Description of Artificial Sequence  Synthetic Peptide
CAVGGGARLMF

SEQ ID NO: 6 - DNA, 33 bp, Artificial Sequence
  Description of Artificial Sequence  Synthetic Primer
tgtgctgtgggtggaggtgccagactcatgttt

SEQ ID NO: 7 - protein, 12 aa, Artificial Sequence
  Description of Artificial Sequence  Synthetic Peptide
CASSGEAFYEQY

SEQ ID NO: 8 - DNA, 36 bp, Artificial Sequence
  Description of Artificial Sequence  Synthetic Primer
tgcgccagcagcggggaggctttctacgagcagtac

SEQ ID NO: 9 - protein, 6 aa, Artificial Sequence
  Description of Artificial Sequence  Synthetic Peptide
TSGFNG

SEQ ID NO: 10 - DNA, 18 bp, Artificial Sequence
  Description of Artificial Sequence  Synthetic Primer
acatctgggttcaacggg

SEQ ID NO: 11 - protein, 5 aa, Artificial Sequence
  Description of Artificial Sequence  Synthetic Peptide
SGHRS

SEQ ID NO: 12 - DNA, 15 bp, Artificial Sequence
  Description of Artificial Sequence  Synthetic Primer
tctggagacctctct

SEQ ID NO: 13 - protein, 6 aa, Artificial Sequence
  Description of Artificial Sequence  Synthetic Peptide
YGATPY

SEQ ID NO: 14 - DNA, 18 bp, Artificial Sequence
  Description of Artificial Sequence  Synthetic Primer
tatggggcaacaccttat

SEQ ID NO: 15 - protein, 5 aa, Artificial Sequence
  Description of Artificial Sequence  Synthetic Peptide
SGHRS

SEQ ID NO: 16 - DNA, 15 bp, Artificial Sequence
  Description of Artificial Sequence  Synthetic Primer
tctgggcacaggagt

SEQ ID NO: 17 - protein, 6 aa, Artificial Sequence
  Description of Artificial Sequence  Synthetic Peptide
YNVLDG

SEQ ID NO: 18 - DNA, 18 bp, Artificial Sequence
  Description of Artificial Sequence  Synthetic Primer
tacaatgttctggatggt

SEQ ID NO: 19 - protein, 6 aa, Artificial Sequence
  Description of Artificial Sequence  Synthetic Peptide
YYNGEE

SEQ ID NO: 20 - DNA, 18 bp, Artificial Sequence
  Description of Artificial Sequence  Synthetic Primer
tattataatggagaagag

SEQ ID NO: 21 - protein, 6 aa, Artificial Sequence
  Description of Artificial Sequence  Synthetic Peptide
YFSGDT

SEQ ID NO: 22 - DNA, 18 bp, Artificial Sequence
  Description of Artificial Sequence  Synthetic Primer
tacttttcaggagacact

SEQ ID NO: 23 - protein, 6 aa, Artificial Sequence
  Description of Artificial Sequence  Synthetic Peptide
YFSETQ

SEQ ID NO: 24 - DNA, 18 bp, Artificial Sequence
  Description of Artificial Sequence  Synthetic Primer
tacttcagtgagacacag

SEQ ID NO: 25 - protein, 269 aa, Artificial Sequence
  Description of Artificial Sequence  Synthetic Peptide
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVSRDSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - DNA, 810 bp, Artificial Sequence
  Description of Artificial Sequence  Synthetic Primer
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacctacatttctgtcttacaatgttctggatggtttggaggagaaaggtcgtttttcttcattccttagtcggtctaaagggtacagttacctccttttgaaggagctccagatgaaagactctgcctcttacctctgtgctgtgagcagggattcaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 27 - protein, 274 aa, Artificial Sequence
  Description of Artificial Sequence  Synthetic Peptide
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPSGTGNIQYFGAGTRLSVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAREWGLGRCLEEIR

SEQ ID NO: 28 - DNA, 825 bp, Artificial Sequence
  Description of Artificial Sequence  Synthetic Primer
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccctagcgggacagggaacattcagtacttcggcgccgggacccggctctcagtgctggaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcacgtgagtggggcctggggagatgcctggaggagattaggtga

SEQ ID NO: 29 - protein, 271 aa, Artificial Sequence
  Description of Artificial Sequence  Synthetic Peptide
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGGGARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - DNA, 816 bp, Artificial Sequence
  Description of Artificial Sequence  Synthetic Primer
atgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtggaggtgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 31 - protein, 273 aa, Artificial Sequence
  Description of Artificial Sequence  Synthetic Peptide
MGSRLLCWVLLCLLGAGPVKAGVTQTPRYLIKTRGQQVTLSCSPISGHRSVSWYQQTPGQGLQFLSEYFSETQRNKGNFPGRFSGRQFSNPRSEMNVSTLELGDSALYLCASSGEAFYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAGEWGLRRCLEEIR

SEQ ID NO: 32 - DNA, 822 bp, Artificial Sequence
  Description of Artificial Sequence  Synthetic Primer
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcacaggagtgtatcctggtaccaacagaccccaggacagggccttcagttcctctctgaatacttcagtgagacacagagaaacaaaggaaacttccctggtcgattctcagggcgccagttctctaaccctcgctctgagatgaatgtgagcaccttggagctgggggactcggccctttatctttgcgccagcagcggggaggctttctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctaaggagatgcctggaggagattaggtga

(19) United States
(12) Patent Application Publication
(10) Pub. No.: US 2008/0064631 A1
(43) Pub. Date: Mar. 13, 2008

(54) T-CELL RECEPTORS FOR USE IN DIAGNOSIS AND THERAPY OF CANCERS AND AUTOIMMUNE DISEASE

(76) Inventor: Jeffrey Molldrem, Houston, TX (US)

Correspondence Address: FULBRIGHT & JAWORSKI L.L.P., 600 CONGRESS AVE., SUITE 2400, AUSTIN, TX, 78701, US

(21) Appl. No.: 11/622,723

(22) Filed: Jan. 12, 2007

Related U.S. Application Data
(60) Provisional application No. 60/758,619, filed on Jan. 13, 2006.

(51) Int. Cl. A61K 38/00; C07H 21/04; C07K 16/00; C12N 15/00; C12Q 1/20

(52) U.S. Cl. 514/12; 435/29; 435/320.1; 530/350; 536/23.5

(57) ABSTRACT

The specification describes the sequences for two T-cell receptors—one having a high affinity and one having low affinity for the HLA-A2-restricted peptide PR1. Use of the T-cell receptors and variants thereof in the diagnosis and treatment of cancer and immune-related diseases are also provided.

This application claims benefit of priority to U.S. Provisional Application Ser. No. 60/758,619, filed Jan. 13, 2006, the entire contents of which are hereby incorporated by reference.

The government owns rights in the present invention pursuant to grant number RO1 CA81247-02 from the National Institutes of Health.

### BACKGROUND OF THE INVENTION

1. Field of the Invention

The present invention relates generally to the fields of cancer and immunotherapy. More particularly, it concerns the identification of immunotherapeutic T-cell receptor sequences and the development of reagents for the treatment and prevention of cancer and autoimmune disease.

2. Description of Related Art

The immune system has long been implicated in the control of cancer; however, evidence for specific and efficacious immune responses in human cancer have been lacking. In chronic myelogenous leukemia (CML), either allogeneic bone marrow transplant (BMT) or interferon-α2b (IFN-α2b) therapy have resulted in complete remission, but the mechanism for disease control is unknown and may involve immune antileukemic responses.

Based on evidence in the art, it is thought that lymphocytes play a role in meditating an antileukemia effect. Studies have demonstrated that allogeneic donor lymphocyte infusions (DLI) have been used to treat relapse of myeloid leukemia after allogeneic BMT (Giralt and Kolb, 1996; Kolb and Holler, 1997; Kolb et al., 1995; Kolb et al., 1996; Antin, 1993). Lymphocyte transfusion from the original bone marrow (BM) donor induces both hematological and cytogenetic responses in approximately 70% to 80% of patients with chronic myelocytic leukemia (CML) in chronic phase (CP) (Kolb et al., 1996, Holler, 1997). Remissions after DLI for AML are generally not as durable as those obtained in chronic phase CML, which may reflect the rapid kinetics of tumor growth outpacing the kinetics of the developing immune response. Additionally, most patients with myeloid forms of leukemia will die from the disease unless they can be treated with allogeneic bone marrow transplant, where the associated graft versus leukemia (GVL) effect cures patients. However, graft-versus-host disease (GVHD) and transplant-related toxicity limit this treatment. It is believed that GVL may be separable from GHVD, and that targeting the immune response toward leukemia-associated antigens will allow for the transfer of GVL to patients without GVHD.

Thus, if more antigens (i.e., leukemia antigens or antigens aganist other cancers) could be determined, and if large numbers of the most potent antigen-specific cytotoxic T lymphocytes (CTLs) could be obtained, it would allow for development of leukemia-specific therapies, breast cancer specific therapies, etc. using the antigens as a targets for vaccines or for generating specific T-cells for use in adoptive immunotherapy.

PR1, an HLAA2.1-restricted nonamer derived from proteinase 3 (P3) and elastase, was identified as a leukemia-associated antigen (Molldrem et al., 2000; Molldrem et al., 1996; Molldrem et al., 1997; Molldrem et al., 1999; Molldrem et al., 2003 each incorporated herein by reference in their entirety). The finding that PR1 is a leukemia-associated antigen has been independently confirmed by Burchert et al. (2002) and Scheibenbogen et al. (2002). CTLs that are specific for PR1 kill AML, CML and MDS cells, but not normal bone marrow cells. In a recent phase I/II vaccine study, the PR1 peptide has been administered to patients with AML, CML and MDS, and PR1-specific CTL immunity has been elicited in 47% of patients, and clincial responses have been observed in 26%. Thus, this antigen provides an interesting platform for further investigation into anti-cancer immune responses as well as for the development of new therapeutic agents.

### SUMMARY OF THE INVENTION

Thus, in accordance with the present invention, there is provided a single chain T-cell receptor comprising an α chain comprising a CDR3α-encoding segment comprising SEQ ID NO:1 or 5 and an β chain comprising a CDR3β-encoding segment comprising SEQ ID NO:3 or 7. The receptor may be purified and isolated. The receptor may lack membrane-spanning regions, and may be soluble. The receptor may be fused to a non-T-cell peptide or polypeptide segment, or may be linked to a diagnostic reagent, such as a fluorophore, a chromophore, a dye, a radioisotope, a chemilluminescent molecule, a paramagnetic ion, or a spin-trapping reagent. The receptor may also be linked to a therapeutic reagent, such as a cytokine, a chemotherapeutic, a radiotherapeutic, a hormone, an antibody Fc fragment, a TLR agonist, a CpG-containing molecule, or an immune co-stimulatory molecule. The receptor may further comprise a dimerization or multimerization sequence.

In another embodiment, there is provided a nucleic acid encoding a single chain T-cell receptor comprising an α chain comprising a CDR3α-encoding segment comprising SEQ ID NO:1 or 5 and an β chain comprising a CDR3β-encoding segment comprising SEQ ID NO:3 or 7. The CDR3α-encoding segment may be encoded by SEQ ID NO:2 or 6 and the CDR3β-encoding segment is encoded by SEQ ID NO:4 or 8. The nucleic acid may further comprise a nucleic acid segment encoding a non-T-cell peptide or polypeptide, and/or further comprise a promoter sequence positioned 5′ to the nucleic acid encoding the T-cell receptor. The promoter may be active in eukaryotic cells and/or in prokaryotic cells. The nucleic acid may be located in a replicable vector, such as a non-viral vector or a viral vector. The nucleic acid may further comprise a linker-encoding segment, wherein the linker-encoding segment is located between the CDR3α-encoding segment and the CDR3β-encoding segment, such as one that encodes a helix-turn-helix motif. The nucleic acid may also further comprise a segment encoding a dimerization or multimerization sequence.

In still another embodiment, there is provided an artificial T-cell receptor comprising an α chain comprising a CDR3α-encoding segment comprising SEQ ID NO:1 or 5 and an β chain comprising a CDR3β-encoding segment comprising SEQ ID NO:3 or 7, wherein the α and β chains are linked by a synthetic linker. The synthetic linker may be a homobifunctional linker. The receptor may lack membrane-spanning regions, and may be soluble. The receptor may be fused to a non-T-cell peptide or polypeptide segment. The receptor may be linked to a diagnostic reagent, such as a fluorophore, a chromophore, a dye, a radioisotope, a chemilluminescent molecule, a paramagnetic ion, or a spin-trapping reagent. The receptor may be linked to a therapeutic reagent, such as a cytokine, a toxin, a chemotherapeutic, a radiotherapeutic, a hormone, an antibody Fc fragment, a TLR agonist, a CpG-containing molecule, or an immune co-stimulatory molecule. The receptor may further comprise a dimerization or multimerization sequence.

In a further embodiment, there is provided a method of making a recombinant T-cell receptor comprising (a) producing a T-cell α chain comprising a CDR3α-encoding segment comprising SEQ ID NO:1 or 5 in a cell; (b) producing a T-cell β chain comprising a CDR3β-encoding segment comprising SEQ ID NO:3 or 7 in a cell; and (c) admixing the α and β chains under conditions supporting disulfide bond formation. The host cell may be a prokaryotic cell or a eukaryotic cell. The CDR3α-encoding segment may be encoded by SEQ ID NO:2 or 6 and the CDR3β-encoding segment is encoded by SEQ ID NO:4 or 8. The method may further comprise linking the recombinant T-cell receptor to a diagnostic or therapeutic agent, such as a fluorophore, a chromophore, a dye, a radioisotope, a chemilluminescent molecule, a paramagnetic ion, a spin-trapping reagent a cytokine, a toxin, a chemotherapeutic, a radiotherapeutic, a hormone, an antibody Fc fragment, a TLR agonist, a CpG-containing molecule, or an immune co-stimulatory molecule.

In still a further embodiment, there is provided a method of making a recombinant T-cell receptor comprising (a) introducing into a host cell (i) a T-cell α chain comprising a CDR3α-encoding segment comprising SEQ ID NO:1 or 5 in a cell and (ii) a T-cell β chain comprising a CDR3β-encoding segment comprising SEQ ID NO:3 or 7 in a cell; and (b) culturing the host cell under conditions supporting expression of the α and β chains. The CDR3α-encoding segment may be encoded by SEQ ID NO:2 or 6 and the CDR3β-encoding segment is encoded by SEQ ID NO:4 or 8. The method may further comprise the step of linking the recombinant T-cell receptor to a diagnostic or therapeutic agent such as a fluorophore, a chromophore, a dye, a radioisotope, a chemilluminescent molecule, a paramagnetic ion, a spin-trapping reagent a cytokine, a toxin, a chemotherapeutic, a radiotherapeutic, a hormone, an antibody Fc fragment, a TLR agonist, a CpG-containing molecule, or an immune co-stimulatory molecule.

In still yet a further embodiment, there is provided a method of detecting abnormal cells in a sample suspected of containing abnormal cells comprising contacting the sample with a single chain T-cell receptor, an artificial T-cell receptor, or a recombinant T-cell receptor, each as described above. The T-cell receptor may be conjugated to a diagnostic agent, such as a fluorophore, a chromophore, a dye, a radioisotope, a chemilluminescent molecule, a paramagnetic ion, or a spin-trapping reagent. The T-cell receptor may be detected using a secondary binding agent, such as an anti-T-cell receptor antibody. The T-cell receptor may comprise a dimerization or multimerization domain. The sample may be (a) a tumor tissue from head & neck, brain, esophagus, breast, lung, liver, spleen, stomach, small intestine, large intestine, rectum, ovary, uterus, cervix, prostate, testicle or skin tissue, or (b) a fluid such as blood, lymph, urine or nipple aspirate. The sample may be from a resected tumor bed. The method may further comprise making a treatment decision based on the presence, absence or degree of detection. Primary cancer cells, metastatic cancer cells or myeloid dysplastic cells may be detected.

In an additional embodiment, there is provided a method of treating a subject with cancer comprising administering to the subject a single chain T-cell receptor, an artificial T-cell receptor, or a recombinant T-cell receptor, where the T-cell receptor is conjugated to a therapeutic agent, each as described above. The cancer may be a solid tumor, such as a head & neck tumor, a brain tumor, an esophageal tumor, a breast tumor, a lung tumor, a liver tumor, a spleen tumor, and stomach tumor, a small intestinal tumor, a large intestinal tumor, a rectal tumor, an ovarian tumor, a uterine tumor, a cervical tumor, a prostate tumor, a testicular tumor or a skin tumor. The cancer may be a blood cancer, such as a leukemia or lymphoma. The therapeutic agent may be a cytokine, a toxin, a chemotherapeutic, a radiotherapeutic, a hormone, an antibody Fc fragment, a TLR agonist, a CpG-containing molecule, or an immune co-stimulatory molecule. The method may further comprise providing the subject with a second anti-cancer therapy, such as a gene therapy, a chemotherapy, a radiotherapy, a hormone therapy, a toxin therapy or surgery. The T-cell receptor may be administered to the subject more than once. The cancer may be recurrent or metastatic cancer.

In still an additional embodiment, there is provided a method of treating a subject with an autoimmune disease comprising administering to the subject a single chain T-cell receptor, an artificial T-cell receptor, or a recombinant T-cell receptor, each as described above. The autoimmune disease may be Wegener's granulomatosis, Churg-Strauss Syndrome, or systemic small vessel vasculitis. The T-cell receptor may be conjugated to a therapeutic agent, such as a toxin or apoptosis-inducing agent. The method may further comprise providing the subject with a second anti-autoimmune therapy, such as an anti-inflammatory agent. The T-cell receptor may be administered to the subject more than once.

In still another embodiment, there is provided a method of transforming a cell comprising (a) providing a target cell; and (b) contacting the target cell with an expression construct comprising a coding region comprising single-chain T-cell receptor comprising an α chain comprising a CDR3α-encoding segment comprising SEQ ID NO:1 or 5 and an β chain comprising a CDR3β-encoding segment comprising SEQ ID NO:3 or 7, wherein the coding region is under the control of a promoter active in the target cell. The expression construct may be a non-viral expression vector or a viral expression vector, such as an adenoviral vector, a retroviral vector, a lentiviral vector, a papillomaviral vector, a poxyiral vector, a herpesviral vector or an adeno-associated viral vector. The target cell may be a lymphocyte, such as a T lymphocyte or a B lymphocyte. The method may further comprise the step of providing the transformed target cell to a subject, which target cell may be obtained from the subject prior to transforming.

Also provided is a method of treating a subject with a myeloid dysplastic disease comprising administering to the subject a single chain T-cell receptor, an artificial T-cell receptor, or a recombinant T-cell receptor, each as described above, wherein the T-cell receptor is conjugated to a therapeutic agent.

In additional embodiments, there are provided (a) a purified and isolated nucleic acid segment encoding a CDR3α comprising the amino sequence of SEQ ID NO:1, for example, wherein the nucleotide sequence comprises SEQ ID NO:2; or (b) a purified and isolated nucleic acid segment encoding a CDR3α and comprising the amino sequence of SEQ ID NO:3, for example, wherein the nucleotide sequence comprises SEQ ID NO:4; (c) a purified and isolated nucleic acid segment encoding a CDR3α comprising the amino sequence of SEQ ID NO:5, for example, wherein the nucleotide sequence comprises SEQ ID NO:6; (d) a purified and isolated nucleic acid segment encoding a CDR3α and comprising the amino sequence of SEQ ID NO:7, for example, wherein the nucleotide sequence comprises SEQ ID NO:8; a (3) a purified and isolated nucleic acid segment encoding the amino sequence of SEQ ID NOS:1 and 3, or SEQ ID NOS:5 and 7, optionally encoded by SEQ ID NOS: 2 and 4, or SEQ ID NOS: 6 and 8. Also provided are purified and isolated nucleic acid segments comprising other CDRs (CDR1 and CDR3), namely, those encoding SEQ ID NOS: 9, 11, 13, 15, 17, 19, 21 or 23, more particularly, SEQ ID NOS: 10, 12, 14, 16, 18, 20, 22 and 24. Also contemplated are sets of three matched CDRs, namely, SEQ ID NOS: 9, 17 and 1, SEQ ID NOS: 11, 19 and 3, SEQ ID NOS: 13, 21 and 5, and SEQ ID NOS: 15, 23 and 7, as well as the corresponding nucleic acids (SEQ ID NOS: 10, 18 and 2, SEQ ID NOS: 12, 20 and 4, SEQ ID NOS: 14, 22 and 6, and SEQ ID NOS: 16, 24 and 8), which may be advantageously combined with appropriate T cell receptor framework regions. Isolated and purified full length sequences (SEQ ID NOS: 25, 27, 29 and 31) and the nucleic acids encoding them (SEQ ID NOS: 26, 28, 30 and 32) are also provided.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Outline of Methods Used in Cloning High and Low Avidity PR1-CTL from an HLA-A2+ Healthy Donor.

FIG. 2—PR1/HLA-A2 Tetramer Staining. Experiment confirms that high & low avidity clones are PR1-specific.

FIG. 3—Spectratype of High Avidity PR1-CTL Clone (F4) Shows Vα7/Vβ1.

FIG. 4—Spectratype of Low Avidity PR1-CTL Clone (B12) Shows Vα7/Vβ1.

FIG. 5—Anti-Vβ5.1 Antibody Staining of Low Avidity PR1-CTL Clone (B12). Experiment confirms that the cDNA Sequence of TCR-Vβ is from the 5.1 Family.

FIG. 6—Cytotoxicity of Peptide-Pulsed T2 Cells. Experiment confirms that F4 PR1-CTL has higher functional avidity that B12 PR1-CTL.

FIG. 7A—cDNA Sequences of TCR-αβ of High Avidity PR1-CTL. Clone F4 expresses the Vα1/Vβ5.1 heterodimeric TCR. Light colored bases highlight primer regions.

FIG. 7B—cDNA Sequences of TCR-αβ of Low Avidity PR1-CTL Clone B12 expresses the Vα1/Vβ5.1 heterodimeric TCR. Light colored bases highlight primer regions.

FIG. 8A—Amino Acid Sequences of TCR-αβ of High Avidity PR1-CTL Clone F4. Translated bases appear above the cDNA sequences.

FIG. 8B—Amino Acid Sequences of TCR-αβ of Low Avidity PR1-CTL Clone B12. Translated bases appear above the cDNA sequences.

### DESCRIPTION OF ILLUSTRATIVE EMBODIMENTS

### I. The Present Invention

The HLA-A2-restricted peptide PR1 is a leukemia-associated antigen with promising therapeutic potential in the treatment of cancer and autoimmune disease. The inventor now reports on the sequence of two T-cell receptors that bind immunologically to the PR1/HLA-A2 ligand. These receptor sequences, and ones having similar binding specificity, can be used to prepare diagnostic and therapeutic reagents for use in the preparation and treatment of various cancers, particularly leukemias, as well as autoimmune diseases. The details of the invention are provided below.

### II. Definitions

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes, see Paul (1993).

“Human leukocyte antigen” or “HLA” is a human class I or class II major histocompatibility complex (MHC) protein (see, e.g., Stites, 1994).

An “HLA supertype or family,” as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

A “protective immune response” refers to a response to an antigen derived from an infectious agent, a tumor antigen, or a self antigen which prevents or at least partially arrests disease symptoms or progression.

“Purified and isolated” means that a given molecule has been purified and/or isolated away from other molecules as its exists in its original or natural form.

“Abnormal cell” is any cell that is considered to have a characteristic a typical for thaT-cell type, including typical growth in an atypical location or typical action against an atypical target. Such cells include cancer cells, benign hyperplastic or dysplastic cells, inflammatory cells or autoimmune cells.

### III. PR-1-Binding T-Cell Receptors

The T-cell receptor (TcR) on CD8+ cytotoxic T lymphocytes (CTLs) is a disulfide-linked heterodimer composed of unique α and β constituent chains that belong to the immunoglobulin (Ig) supergene family. All CTL precursors, as they mature in the thymus, express a unique TCR-αβ pair, which assembles during thymic development from germ line α and β genes, which are rearranged to form a mature receptor that is expressed on the plasma membrane.

The TcR's ligand, a peptide/MHC-1 complex, is created and expressed on the plasma membrane of the target cell or antigen presenting cell (APC). When bound to cognate peptide/MHC-1 on the target cell, TcR on the CTL transduce a signal to the nucleus which causes the CTL to become activated and to subsequently proliferate and kill the target cell that expresses the peptide/MHC-1 ligand.

The present invention involves the cloning and sequencing of two different PR1-specific CTL clones that were derived from an HLA-A2+ healthy individual. One of these clones bears a relatively high avidity TcR for the PR1/HLA-A2 ligand (SEQ ID NOS:25 and 27), while the other bears a relatively low avidity TcR (SEQ ID NOS:29 and 31). These receptor sequences can be used in a variety of different fashions, as discussed further below.

### IV. Nucleic Acids

One aspect of the TcR segments described above are the nucleic acid segment that encode them. A nucleic acid segment may be derived from genomic DNA, complementary DNA (cDNA) or synthetic DNA. Where incorporation into an expression vector is desired, the nucleic acid may also comprise a natural intron or an intron derived from another gene, as well as other non-coding (e.g., regulatory) and coding regions (e.g., linkers). As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein.

The term “recombinant” may be used in conjunction with a polypeptide or the name of a specific polypeptide, and generally refers to a polypeptide produced from a nucleic acid molecule that has been manipulated in vitro or that is the replicated product of such a molecule. Recombinant vectors and isolated nucleic acid segments may variously include the PR1-coding regions themselves, coding regions bearing selected alterations or modifications in the basic coding region, or they may encode larger polypeptides that nevertheless include PR1-coding regions or may encode biologically functional equivalent proteins or peptides that have variant amino acids sequences.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length.

It is contemplated that the TcR may be encoded by any nucleic acid sequence that encodes the appropriate amino acid sequence, and not only those exemplified by SEQ ID NOS: 26, 28, 30, and 32. The design and production of nucleic acids encoding a desired amino acid sequence is well known to those of skill in the art, using standardized codon tables (Table 1). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et al., 1990). Codon preferences for other organisms also are well known to those of skill in the art (Wada et al., 1990, included herein in its entirety by reference).

TABLE 1
Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

### V. T-Cell Receptors

T-cell receptors (TcRs) are heterodimeric molecules containing α and β chains. The TcR chains are organized much like Ig chains. Their N-terminal portions are variable and their C-terminal portions are constant. The variable (V) region of the TcR-β chain is encoded by a gene made of 3 distinct genetic elements (Vβ, D and Jb) that are separated in the germline configuration. The TcRα chain follows similarly, but it does not use a D gene. In the V-region domains of each chain, there are 3 complementarity-determining regions (CDRs, numbered 1 to 3), and the six combined CDRs from the two variable domains (α and β) form the antigen-binding surface of the TcR. The CDR3 region encodes the portion of the TCR chains that binds to the cognate peptide in the groove of the MHC-1 chains, and between the CDR3 loops of the Vα and Vβ, there is a pocket that accommodates side chains from the peptide bound to the MHC-1.

As discussed, the present invention relates to TcR that bind to PR1 peptides. The sequences of the α and β chains for two distinct TcR are set forth in SEQ ID NOS:25-32 are exemplary of PR1-binding TcR. However, various other amino acid sequences can provide the same PR1-binding activity. Such sequences may differ in some positions from the sequences provided above by substituting one or more amino acid molecules in the given sequence. The substitutions are preferably conservative, i.e., the maintain the same charge and structure of the substituted residue, and normally are limited to a few residues (1, 2, 3, 4, 5, 6, 7, 8, 9 or 10 residues within a binding pocket). Another reason that one may alter residues of the TcR of the present invention is to alter the binding affinity. The full length TcR chain represented by SEQ ID NOS:25 and 27 is of higher affinity than that of SEQ ID NOS:29 and 31, and thus one may prefer to select one over the other for different uses (diagnostic versus therapeutic; cancer therapeutics verus immune therapeutics). The following table lists the various SEQ ID NOS of this application.

TABLE 2
SEQ ID NO ASSIGNMENTS

| SEG- | | | | | |
|---|---|---|---|---|---|
| MENT | | | | | |
| (Hi | NUCLEIC | | SEGMENT | NUCLEIC | |
| Affinity) | ACID | PROTEIN | (Lo Affinity) | ACID | PROTEIN |
| Full | 26 | 25 | Full | 30 | 29 |
| Length α | | | Length α | | |
| CD1Rα | 10 | 9 | CDR1α | 14 | 13 |
| CD2Rα | 18 | 17 | CDR2α | 22 | 21 |
| CD3Rα | 2 | 1 | CDR3α | 6 | 6 |
| Full | 28 | 27 | Full | 32 | 31 |
| Length β | | | Length β | | |
| CD1Rβ | 12 | 11 | CDR1β | 16 | 15 |
| CD2Rβ | 20 | 19 | CDR2β | 24 | 23 |
| CD3Rβ | 4 | 3 | CDR3β | 8 | 7 |

The following is a discussion based upon changing the amino acids of a protein to create a modified protein. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention may also employ the use of peptide mimetics for the preparation of polypeptides (see e.g., Johnson, 1993) having many of the natural properties of a TcR, but with altered and/or improved characteristics. The underlying rationale behind the use of mimetics is that the peptide backbone of proteins exists chiefly to orient amino acid side chains in such a way as to facilitate molecular interactions, such as those of antibody and antigen. These principles may be used, in conjunction with the principles outline above, to engineer second generation molecules having many of the natural properties of a TcR but with altered and even improved characteristics.

It is contemplated that the present invention may further employ sequence variants such as insertional or deletion variants. Deletion variants lack one or more residues of the native protein. Insertional mutants typically involve the addition of material at a non-terminal point in the polypeptide. It also will be understood that insertional sequence variants may include N- or C-terminal amino acids, and yet still be essentially as set forth in one of the sequences disclosed herein, so long as the sequence meets the criteria set forth above, including the maintenance of biological activity.

As used herein, an “amino acid” or “amino acid residue” refers to any naturally-occurring amino acid, any amino acid derivative or any amino acid mimic known in the art, including modified or unusual amino acids. In certain embodiments, the residues of the TcR sequential, without any non-amino acid interrupting the sequence of amino acid residues. In other embodiments, the sequence may comprise one or more non-amino acid moieties. In particular embodiments, the sequence of residues of the TcR may be interrupted by one or more non-amino acid moieties.

Accordingly, the term “proteinaceous composition” encompasses amino molecule sequences comprising at least one of the 20 common amino acids in naturally-synthesized proteins, or at least one modified or unusual amino acid.

TcR may be made by any technique known to those of skill in the art, including expression through standard molecular biological techniques, or the chemical synthesis of polypeptides. Methods for recombinant expression are addressed elsewhere in this document.

A. Recombinant Single Chain TcR Single chain TcRs are designed and produced much the same way as single chain antibodies. In short, the α chain C and V regions of the TcR are cloned into a single expression vector under the control of a promoter region that is capable of directing the expression of the chain. Then, the β chain variable region is added to the Vα region by use of a flexible linker, such that the VP region can fold back and form an antigen biding pocket in conjunction with the Vα region.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant T-cell receptors of the present invention may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two T-cell receptors.

B. Synthetic Single Chain TcR

In a separate embodiment, a single-chain TcR can be created by joining receptor light and heavy chains using a non-peptide linker or chemical unit. Generally, the light and heavy chains will be produced in distincT-cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie together functional groups of two different molecules, e.g., a stabilizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation. Table 3 illustrates several of these cross-linkers.

TABLE 3
HETERO-BIFUNCTIONAL CROSS-LINKERS

| | | | Spacer Arm | |
|---|---|---|---|---|
| | | | Length\after cross- | |
| linker | Reactive Toward | Advantages and Applications | linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC-SPDP | Primary amines | Extended spacer arm | 15.6 | A |
| | Sulfhydryls | Water-soluble | | |
| SMCC | Primary amines | Stable maleimide reactive group | 11.6 | A |
| | Sulfhydryls | Enzyme-antibody conjugation | | |
| | | Hapten-carrier protein conjugation | | |
| Sulfo-SMCC | Primary amines | Stable maleimide reactive group | 11.6 | A |
| | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein conjugation | | |
| Sulfo-MBS | Primary amines | Water-soluble | 9.9 | A |
| | Sulfhydryls | | | |
| SIAB | Primary amines | Enzyme-antibody conjugation | 10.6 | A |
| | Sulfhydryls | | | |
| Sulfo-SIAB | Primary amines | Water-soluble | 10.6 | A |
| | Sulfhydryls | | | |
| SMPB | Primary amines | Extended spacer arm | 14.5 | A |
| | Sulfhydryls | Enzyme-antibody conjugation | | |
| Sulfo-SMPB | Primary amines | Extended spacer arm | 14.5 | A |
| | Sulfhydryls | Water-soluble | | |
| EDC/Sulfo-NHS | Primary amines | Hapten-Carrier conjugation | 0 | |
| | Carboxyl groups | | | |
| ABH | Carbohydrates | Reacts with sugar groups | 11.9 | A |
| | Nonselective | | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

C. TcR Purification

In certain embodiments, the TcRs of the present invention may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur.

Generally, “purified” also refers to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying a TcR of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

D. Conjugation of TcR to Therapeutic or Diagnostic Agents

In one embodiment, the TcRs of the present invention may be linked to various reagents for use in diagnosis and therapy of disease. Linking may be performed using a variety of well known chemical reactions and agents, some of which are described elsewhere in this document.

1. Diagnostic Reagents

Many diagnostic/imaging agents are known in the art, as are methods for their attachment to proteins (see, for e.g., U.S. Pat. Nos. 5,021,236; 4,938,948; and 4,472,509, each incorporated herein by reference). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶-chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled receptors of the present invention may be produced according to well-known methods in the art. For instance, receptors can be iodinated by contact with sodium and/or potassium iodide and a chemical oxidizing agent such as sodium hypochlorite, or an enzymatic oxidizing agent, such as lactoperoxidase. TcRs according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes, which exist as metallic ions to antibody are diethylenetriaminepentaacetic acid (DTPA) or ethylene diaminetetracetic acid (EDTA).

Among the fluorescent labels contemplated for use as conjugates are Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

2. Therapeutic Reagents

A wide variety of therapeutic agents made linked to TcR of the present invention. For example, the radioisotopes discussed above, though useful in diagnostic contexts, may be also be used as therapeutic agents. Chemotherapeutics may also be conjugated to TcR, and include cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate.

Another class of therapeutic agent is the toxins. Cholera toxin, botulism toxin, pertussis toxin, ricin A and B chains, as well as other natural or synthetic toxins are contemplated.

Cytokines and lymphokines are yet another class of therapeutic agents than can be coupled to the TcR of the present invention, and include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, TNFα, GM-CSF, INFα, IFNβ, and IFNγ.

In other embodiments, anti-inflammatory agents are contemplated as therapeutic agents that may be conjugated to TcR. Anti-inflammatories include NSAIDs, steroids, rapamycin, infliximab, and ontak. Immunosuppressive agents include FK-506 and cyclosporin A.

### VI. Administration of TcR for Diagnosis of Cancer or Hyperplastic or Dysplastic Disorders

In an embodiment of the present invention, there are provided methods of diagnosing cancers such as leukemia (e.g., AML, CML, MDS), as well as myelodysplastic disorders. Myelodysplasias (MDS) refer to a group of disorders in which the bone marrow does not function normally and produces insufficient number of normal blood cells. MDS affects the production of any, and occasionally all, types of blood cells including red blood cells, platelets, and white blood cells (cytopenias). About 50 percent of pediatric myelodysplasia can be classified in five types of MDS: refractory anemia, refractory anemia with ring sideroblasts, refractory anemia with excess blasts, refractory anemia with excess blasts in transformation, and chronic myelomonocytic leukemia. The remaining 50 percent typically present with isolated or combined cytopenias such as anemia, leucopenia and/or thrombocytopenia (low platelet count). Although chronic, MDS progresses to become acute myeloid leukemia (AML) in about 30 percent of patients.

Also contemplated for diagnosis according to the present invention are solid tumor cancers. Such cancer lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. Other hyperplastic, neoplastic and dysplastic diseases, including benign hyperprolifertative diseases are also with the scope of the diagnostic procedures described herein.

A. Administration of Diagnostic Reagents

Administration of diagnostic reagents is well known in the art and will vary depending on diagnosis to be achieved. For example, where a discrete tumor mass or masses is/are to be imagined, local or regional administration (e.g., in the tumor vasculature, local lymph system or local arteries or veins) my be utilized. Alternatively, one may provide diagnostic reagents regionally or systemically. This may be the route of choice where imaging of an entire limb or organism is desired, where know specific tumor mass has been identified, or when metastasis is suspected.

B. Injectable Compositions and Formulations

One method for the delivery of a pharmaceutical according to the present invention is systemically. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of pharmaceuticals may be by syringe or any other method used for injection of a solution, as long as the agent can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### VII. Therapeutic Methods

A. Cancer and Hyperplastic/Dysplastic/Neoplastic Disease

The TcR of the present invention may be used in the methods of treating hyperplastic/dysplastic/neoplastic diseases/conditions including cancer. Types of diseases/conditions contemplated to be treated with the peptides of the present invention include, but are not limited to leukemias such as, AML, MDS and CML, as well as myelodysplasias. Other types of cancers may include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, bladder cancer and any other neoplastic diseases.

To kill cells, inhibit-cell growth, inhibit metastasis, decrease tumor or tissue size and otherwise reverse or reduce the malignant phenotype of tumor cells, using the methods and compositions of the present invention, one would generally contact a cancer cell with the therapeutic compound such as a polypeptide or an expression construct encoding a polypeptide. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Any of the formulations and routes of administration discussed with respect to the treatment or diagnosis of cancer may also be employed with respect to neoplastic diseases and conditions. Ex vivo embodiments, where tumor cells are treated/transduced outside a patient's body (either specifically or as part of a larger cell population) are contemplated.

Adoptive immunotherapy is also contemplated as an embodiment of treating cancer according to the present invention. T cells, in particular CD8⁺ T cells, can be transduced with the high avidity TcR of the present invention, and subsequently are introduced (or reintroduced) into a subject. Such T cells will be rendered more effective at responding to the cancer cells, in particular, leukemia cells, CLL cells, AML cells and MDS cells.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used may be used at the time of surgery, and/or thereafter, to treat residual or metastatic disease. For example, a resected tumor bed may be injected or perfused with a formulation comprising a tumor-associated HLA restricted peptide or construct encoding therefor. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hr, to about 2-6 hr, to about 6-12 hr, to about 12-24 hr, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6-dose application over a two-week period. The two-week regimen may be repeated one, two, three, four, five, six or more times. During a course of treatment, the need to complete the planned dosings may be re-evaluated.

B. Combination Therapies

It also may prove advantageous to use combination therapies, where a second anti-cancer agent is included. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided with a therapy according to the present invention in a combined amount effective to kill or inhibit proliferation of the cell. This process may involve contacting the cells with the both agent(s) at the same time. This may be achieved by contacting the cell with a single composition or pharmacological formulation that includes both agents, or by contacting the cell with two distinct compositions or formulations at the same time.

Alternatively, the TcR receptor therapy may precede or follow the other agent treatment by intervals ranging from minutes to weeks. In embodiments where the other agent and TcR are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed; for example, the TcR (with or without a conjugated therapeutic agent) or TcR expression construct is “A” and the secondary anti-cancer therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic agents of the present invention to a patient will follow general protocols for the administration of that particular secondary therapy, taking into account the toxicity, if any, of the TcR treatment. It is expected that the treatment cycles would be repeated as necessary. It also is contemplated that various standard therapies, as well as surgical intervention, may be applied in combination with the described cancer therapies.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

2. Radiotherapy

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effecT-cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons, and; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

Adoptive Immunotherapy. In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

Passive Immunotherapy. A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989). Possible therapeutic antibodies include anti-TNF, anti-CD25, anti-CD3, anti-CD20, CTLA-4-IG, and anti-CD28.

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

4. Gene Therapy

In yet another embodiment, the secondary treatment is a gene therapy in which a therapeutic polynucleotide is administered before, after, or at the same time as the tumor-associated HLA-restricted peptide is administered. Delivery of a vector encoding a the tumor-associated HLA-restricted peptide in conjunction with a second vector encoding one of the following gene products will have a combined anti-hyperproliferative effect on target tissues. Alternatively, a single vector encoding both genes may be used. A variety of proteins are encompassed within the invention, some of which are described below. Various genes that may be targeted for gene therapy of some form in combination with the present invention are will known to one of ordinary skill in the art and may comprise any gene involved in cancers.

Inducers of Cellular Proliferation. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity. The proteins Jun, Fos and Myc are proteins that directly exert their effects on nuclear functions as transcription factors.

Inhibitors of Cellular Proliferation. The tumor suppressor oncogenes function to inhibit excessive cellular proliferation. The inactivation of these genes destroys their inhibitory activity, resulting in unregulated proliferation. The tumor suppressors p53, p16 and C-CAM are described below.

In addition to p53, which has been described above, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Regulators of Programmed Cell Death. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

C. Autoimmune Diseases

1. Vasculitis

Vasculitis is a process caused by inflammation of blood vessel walls and results in a variety of disorders. An accepted classification system for vasculitis has not emerged, although it may be categorized by the size or type of the involved blood vessel as large-, medium-, or small-vessel vasculitis. Small-vessel vasculitis is defined as vasculitis that affects vessels smaller than arteries (i.e., arterioles, venules, and capillaries); however, small-vessel vasculitis can also involve medium-sized arteries. Anti-neutrophil cytoplasmic antibodies (ANCA)-associated vasculitis is the most common cause of small-vessel vasculitis and includes microscopic polyangiitis, Wegener's granulomatosis, Churg-Strauss syndrome, and certain types of drug-induced vasculitis.

Wegener's Granulomatosis. Wegener's Granulomatosis is a rare disorder which causes inflammation of blood vessels in the upper respiratory tract (nose, sinuses, ears), lungs, and kidneys. Many other areas of the body may also be affected, with arthritis (joint inflammation) occurring in almost half of all cases. The eyes and skin may also be affected. The cause is unknown, but Wegener's Granulomatosis is thought to be an autoimmune disorder and is often classified as one of the rheumatic diseases. Destructive lesions develop in the upper and lower respiratory tract and the kidney. In the kidney, these lesions cause glomerulonephritis that may result in hematuria (blood in the urine) and kidney failure. It occurs most often between the ages of 30 and 50, and men are affected twice as often as women. It is rare in children, but has been seen in infants as young as 3 months old. The kidney disease can progress rapidly, with kidney failure occurring within months of the initial diagnosis. If untreated, kidney failure and death occur in more than 90% of all patients with Wegener's granulomatosis.

Early symptoms may include fatigue, malaise, fever, and a sense of discomfort around the nose and sinuses. Upper respiratory infections such as sinusitis or ear infections frequently precede the diagnosis of Wegener's Granulomatosis. Other upper respiratory symptoms include nose bleeds, pain, and sores around the opening of the nose. Persistent fever without an obvious cause (fever of undetermined origin—FUO) may be an initial symptom. Night sweats may accompany the fever. Loss of appetite and weight loss are common. Skin lesions are common, but there is no one characteristic lesion associated with the disease. Kidney disease is necessary to make the definitive diagnosis of Wegener's Granulomatosis. The urine may be bloody, which often first appears as red or smoky urine. There may be no symptoms, but is easily diagnosed with laboratory studies. Eye problems develop in a significant number of patients and may range from a mild conjunctivitis to severe inflammation of the eyeball and the tissues around the eyeball. Additional symptoms include weakness, loss of appetite, weight loss, bloody discharge from the nose, pain over the sinuses, sinusitis, lesions in and around the opening of the nose, cough, coughing up blood, bloody sputum, shortness of breath, wheezing, chest pain, blood in the urine, rashes, and joint pain.

Diagnosis as made by take a biopsy of abnormal tissue, which may include open lung biopsy, upper airway biopsy, nasal mucosal biopsy, bronchoscopy with transtracheal biopsy, kidney biopsy, urinalysis, chest x-ray, bone marrow aspiration, blood test (for autoantibodies). Treatment includes corticosteroids, cyclophosphamide, methotrexate, or azathioprine, which can produce long-term remission in over 90% of affected people.

Churg-Strauss Syndrome. Churg-Strauss Syndrome (CSS), also known as allergic granulomatosis, is a form of systemic vasculitis. CSS is similar to polyarteritis nodosa, but the abundance of eosinophils distinguished this disease. Most CSS patients are middle-aged, with a history of new or increased severity asthma—asthma being one of the cardinal features of CSS. The symptoms of asthma may begin long before the onset of vasculitis. Other early symptoms include nasal polyps and allergic rhinitis. The disease often transitions into eosinophilia, with counts reaching as high as 60%. The next phase of disease is overt vasculitis, which can involve the skin, lungs, nerves, kidneys, and other organs. Peripheral nerve involvement can be particularly debilitating and includes pain, numbness, or tingling in extremities (neuropathy/mononeuritis multiplex). Prior to the advent of therapies, CSS was often a fatal disease. The majority of patients died from rampant, uncontrolled disease.

The cause of CSS is not known, but it is like multi-factorial. Though a genetic factor may exist, CSS is only rarely seen in two members of the same family. Thus, environmental factors and infections are more likely to be the cause, but there is no definitive evidence of this. Diagnosis is performed by a specific combination of symptoms and signs, the pattern of organ involvement, and the presence of certain abnormal blood tests (eosinophilia, in particular). In addition to a detailed patient history and physical examination, blood tests, chest X-rays and other types of imaging studies, nerve conduction tests, and tissue biopsies (lung, skin, or nerve) may be performed to aid in the diagnosis. In order to be classified as a CSS patient, a patient should have at least 4 of the following 6 criteria: 1) asthma; 2) eosinophilia [>10% on differential WBC count]; 3) mononeuropathy; 4) transient pulmonary infiltrates on chest X-rays; 5) paranasal sinus abnormalities; and 6) biopsy containing a blood vessel with extravascular eosinophils.

CSS usually responds to prednisone. Initially, high doses of oral prednisone are used, but after the first month or so, this high dose of prednisone is gradually tapered down over the ensuing months. Other immunosuppressive drugs, such as azathioprine, cellcept, methotrexate, or cyclophosphamide may be used in addition to prednisone. High doses of intravenous steroids maybe useful for those patients with severe disease, or for those who are unresponsive to other treatments. With proper therapy, symptoms begin to resolve quickly, with gradual improvement in cardiac and renal function, as well as improvement in the pain that results from peripheral nerve involvement. Therapy may last for 1 to 2 years, depending on patient response and continuation of disease.

2. Crohn's Disease

Crohn's disease symptoms include intestinal inflammation and the development of intestinal stenosis and fistulas; neuropathy often accompanies these symptoms. Anti-inflammatory drugs, such as 5-aminosalicylates (e.g., mesalamine) or corticosteroids, are typically prescribed, but are not always effective (reviewed in V. A. Botoman et al., 1998). Immunosuppression with cyclosporine is sometimes beneficial for patients resistant to or intolerant of corticosteroids (Brynskov et al., 1989).

Nevertheless, surgical correction is eventually required in 90% of patients; 50% undergo colonic resection (Leiper et al., 1998; Makowiec et al., 1998). The recurrence rate after surgery is high, with 50% requiring further surgery within 5 years (Leiper et al., 1998; Besnard et al., 1998).

One hypothesis for the etiology of Crohn's disease is that a failure of the intestinal mucosal barrier, possibly resulting from genetic susceptibilities and environmental factors (e.g., smoking), exposes the immune system to antigens from the intestinal lumen including bacterial and food antigens (e.g., Soderholm et al., 1999; Hollander et al., 1986; D. Hollander, 1992). Another hypothesis is that persistent intestinal infection by pathogens such as Mycobacterium paratuberculosis, Listeria monocytogenes, abnormal Escherichia coli, or paramyxovirus, stimulates the immune response; or alternatively, symptoms result from a dysregulated immune response to ubiquitous antigens, such as normal intestinal microflora and the metabolites and toxins they produce (Sartor, 1997). The presence of IgA and IgG anti-Sacccharomyces cerevisiae antibodies (ASCA) in the serum was found to be highly diagnostic of pediatric Crohn's disease (Ruemmele et al., 1998; Hoffenberg et al., 1999).

In Crohn's disease, a dysregulated immune response is skewed toward cell-mediated immunopathology (Murch, 1998). But immunosuppressive drugs, such as cyclosporine, tacrolimus, and mesalamine have been used to treat corticosteroid-resistant cases of Crohn's disease with mixed success (Brynskov et al., 1989; Fellerman et al., 1998).

Recent efforts to develop diagnostic and treatment tools against Crohn's disease have focused on the central role of cytokines (Schreiber, 1998; van Hogezand & Verspaget, 1998). Cytokines are small, secreted proteins or factors (5 to 20 kD) that have specific effects on cell-to-cell interactions, intercellular communication, or the behavior of other cells. Cytokines are produced by lymphocytes, especially T_{H}1 and T_{H}2 lymphocytes, monocytes, intestinal macrophages, granulocytes, epithelial cells, and fibroblasts (reviewed in Rogler & Andus, 1998; Galley & Webster, 1996). Some cytokines are pro-inflammatory (e.g., TNF-α, IL-1(α and β), IL-6, IL-8, IL-12, or leukemia inhibitory factor (LIF)); others are anti-inflammatory (e.g., IL-1 receptor antagonist, IL-4, IL-10, IL-11, and TGF-β). However, there may be overlap and functional redundancy in their effects under certain inflammatory conditions.

In active cases of Crohn's disease, elevated concentrations of TNF-α and IL-6 are secreted into the blood circulation, and TNF-α, IL-1, IL-6, and IL-8 are produced in excess locally by mucosal cells (Funakoshi et al., 1998). These cytokines can have far-ranging effects on physiological systems including bone development, hematopoiesis, and liver, thyroid, and neuropsychiatric function. Also, an imbalance of the IL-1β/IL-1ra ratio, in favor of pro-inflammatory IL-1β, has been observed in patients with Crohn's disease (Rogler & Andus, 1998; Saiki et al., 1998; Dionne et al., 1998; but see S. Kuboyama, 1998). One study suggested that cytokine profiles in stool samples could be a useful diagnostic tool for Crohn's disease (Saiki et al., 1998).

Treatments that have been proposed for Crohn's disease include the use of various cytokine antagonists (e.g., IL-1ra), inhibitors (e.g., of IL-1β converting enzyme and antioxidants) and anti-cytokine antibodies (Rogler and Andus, 1998; van Hogezand & Verspaget, 1998; Reimund et al., 1998; N. Lugering et al., 1998; McAlindon et al., 1998). In particular, monoclonal antibodies against TNF-α have been tried with some success in the treatment of Crohn's disease (Targan et al., 1997; Stack et al., 1997; van Dullemen et al., 1995). These compounds can be used in combination therapy with compounds of the present invention.

Another approach to the treatment of Crohn's disease has focused on at least partially eradicating the bacterial community that may be triggering the inflammatory response and replacing it with a non-pathogenic community. For example, U.S. Pat. No. 5,599,795 discloses a method for the prevention and treatment of Crohn's disease in human patients. Their method was directed to sterilizing the intestinal tract with at least one antibiotic and at least one anti-fungal agent to kill off the existing flora and replacing them with different, select, well-characterized bacteria taken from normal humans. Borody taught a method of treating Crohn's disease by at least partial removal of the existing intestinal microflora by lavage and replacement with a new bacterial community introduced by fecal inoculum from a disease-screened human donor or by a composition comprising Bacteroides and Escherichia coli species (U.S. Pat. No. 5,443,826). However, there has been no known cause of Crohn's disease to which diagnosis and/or treatment could be directed.

3. Rheumatoid Arthritis

The exact etiology of RA remains unknown, but it is clear that it has autoimmune aspects. The first signs of joint disease appear in the synovial lining layer, with proliferation of synovial fibroblasts and their attachment to the articular surface at the joint margin (Lipsky, 1998). Subsequently, macrophages, T-cells and other inflammatory cells are recruited into the joint, where they produce a number of mediators, including the cytokines interleukin-1 (IL-1), which contributes to the chronic sequalae leading to bone and cartilage destruction, and tumour necrosis factor (TNF-α), which plays a role in inflammation (Dinarello, 1998; Arend & Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger & Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1ra), a member of the IL-1 gene family (Hannum et al., 1990). IL-1ra, as its name indicates, is a natural receptor antagonist that competes with IL-1 for binding to type I IL-1 receptors and, as a result, blocks the effects of IL-1 (Arend et al., 1998). A 10- to 100-fold excess of IL-1ra may be needed to block IL-1 effectively; however, synovial cells isolated from patients with RA do not appear to produce enough IL-1ra to counteract the effects of IL-1 (Firestein et al., 1994; Fujikawa et al., 1995).

4. Systemic Lupus Erythematosus

Systemic lupus erythematosus (SLE) is an autoimmune rheumatic disease characterized by deposition in tissues of autoantibodies and immune complexes leading to tissue injury (Kotzin, 1996). In contrast to autoimmune diseases such as MS and type 1 diabetes mellitus, SLE potentially involves multiple organ systems directly, and its clinical manifestations are diverse and variable (reviewed by Kotzin & O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996).

The serological hallmark of SLE, and the primary diagnostic test available, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of lupus glomerulonephritis (G N) (Hahn & Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

The mechanisms by which autoantibodies are induced in these autoimmune diseases remain unclear. As there has been no known cause of SLE, to which diagnosis and/or treatment could be directed, treatment has been directed to suppressing immune responses, for example with macrolide antibiotics, rather than to an underlying cause. (e.g., U.S. Pat. No. 4,843,092).

4. Juvenile Rheumatoid Arthritis

Juvenile rheumatoid arthritis (JRA), a term for the most prevalent form of arthritis in children, is applied to a family of illnesses characterized by chronic inflammation and hypertrophy of the synovial membranes. The term overlaps, but is not completely synonymous, with the family of illnesses referred to as juvenile chronic arthritis and/or juvenile idiopathic arthritis in Europe.

Jarvis (1998) and others (Arend, 2001) have proposed that the pathogenesis of rheumatoid disease in adults and children involves complex interactions between innate and adaptive immunity. This complexity lies at the core of the difficulty of unraveling disease pathogenesis.

Both innate and adaptive immune systems use multiple cell types, a vast array of cell surface and secreted proteins, and interconnected networks of positive and negative feedback (Lo et al., 1999). Furthermore, while separable in thought, the innate and adaptive wings of the immune system are functionally intersected (Fearon & Locksley, 1996), and pathologic events occurring at these intersecting points are likely to be highly relevant to our understanding of pathogenesis of adult and childhood forms of chronic arthritis (Warrington, et al., 2001).

Polyarticular JRA is a distinct clinical subtype characterized by inflammation and synovial proliferation in multiple joints (four or more), including the small joints of the hands (Jarvis, 2002). This subtype of JRA may be severe, because of both its multiple joint involvement and its capacity to progress rapidly over time. Although clinically distinct, polyarticular JRA is not homogeneous, and patients vary in disease manifestations, age of onset, prognosis, and therapeutic response. These differences very likely reflect a spectrum of variation in the nature of the immune and inflammatory attack that can occur in this disease (Jarvis, 1998).

5. Sjögren's Syndrome

Primary Sjögren's syndrome (SS) is a chronic, slowly progressive, systemic autoimmune disease, which affects predominantly middle-aged women (female-to-male ratio 9:1), although it can be seen in all ages including childhood (Jonsson et al., 2002). It is characterized by lymphocytic infiltration and destruction of the exocrine glands, which are infiltrated by mononuclear cells including CD4+, CD8+ lymphocytes and B-cells (Jonsson et al., 2002). In addition, extraglandular (systemic) manifestations are seen in one-third of patients (Jonsson et al., 2001).

The glandular lymphocytic infiltration is a progressive feature (Jonsson et al., 1993), which, when extensive, may replace large portions of the organs. Interestingly, the glandular infiltrates in some patients closely resemble ectopic lymphoid microstructures in the salivary glands (denoted as ectopic germinal centers) (Salomonsson et al., 2002; Xanthou & Polihronis, 2001). In SS, ectopic GCs are defined as T and B cell aggregates of proliferating cells with a network of follicular dendritic cells and activated endothelial cells. These GC-like structures formed within the target tissue also portray functional properties with production of autoantibodies (anti-Ro/SSA and anti-La/SSB) (Salomonsson &, Jonsson, 2003).

In other systemic autoimmune diseases, such as RA, factors critical for ectopic GCs have been identified. Rheumatoid synovial tissues with GCs were shown to produce chemokines CXCL13, CCL21 and lymphotoxin (LT)-β (detected on follicular center and mantle zone B cells). Multivariate regression analysis of these analytes identified CXCL13 and LT-β as the solitary cytokines predicting GCs in rheumatoid synovitis (Weyand & Goronzy, 2003). Recently CXCL13 and CXCR5 in salivary glands has been shown to play an essential role in the inflammatory process by recruiting B and T-cells, therefore contributing to lymphoid neogenesis and ectopic GC formation in SS (Salomonsson & Larsson, 2002).

6. Psoriasis

Psoriasis is a chronic skin disease of scaling and inflammation that affects 2 to 2.6 percent of the United States population, or between 5.8 and 7.5 million people. Although the disease occurs in all age groups, it primarily affects adults. It appears about equally in males and females. Psoriasis occurs when skin cells quickly rise from their origin below the surface of the skin and pile up on the surface before they have a chance to mature. Usually this movement (also called turnover) takes about a month, but in psoriasis it may occur in only a few days. In its typical form, psoriasis results in patches of thick, red (inflamed) skin covered with silvery scales. These patches, which are sometimes referred to as plaques, usually itch or feel sore. They most often occur on the elbows, knees, other parts of the legs, scalp, lower back, face, palms, and soles of the feet, but they can occur on skin anywhere on the body. The disease may also affect the fingernails, the toenails, and the soft tissues of the genitals and inside the mouth. While it is not unusual for the skin around affected joints to crack, approximately 1 million people with psoriasis experience joint inflammation that produces symptoms of arthritis. This condition is called psoriatic arthritis.

Psoriasis is a skin disorder driven by the immune system, especially involving T-cells. In psoriasis, T-cells are put into action by mistake and become so active that they trigger other immune responses, which lead to inflammation and to rapid turnover of skin cells. In about one-third of the cases, there is a family history of psoriasis. Researchers have studied a large number of families affected by psoriasis and identified genes linked to the disease. People with psoriasis may notice that there are times when their skin worsens, then improves. Conditions that may cause flareups include infections, stress, and changes in climate that dry the skin. Also, certain medicines, including lithium and betablockers, which are prescribed for high blood pressure, may trigger an outbreak or worsen the disease.

7. Multiple Sclerosis

Multiple sclerosis (MS) continues to be a serious health problem that afflicts hundreds of thousands each year in the US alone, and millions worldwide. It is one of the most common diseases of the central nervous system (brain and spinal cord). MS is an inflammatory condition associated with demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord, hence the term “multiple” sclerosis, literally meaning many scars.

Currently, there is no single laboratory test, symptom, or physical finding that provides a conclusive diagnosis of MS. To complicate matters, symptoms of MS can easily be confused with a wide variety of other diseases such as acute disseminated encephalomyelitis, Lyme disease, HIV-associated myelopathy, HTLV-I-associated myelopathy, neurosyphilis, progressive multifocal leukoencephalopathy, systemic lupus erythematosus, polyarteritis nodosa, Sjögren's syndrome, Behçet's disease, sarcoidosis, paraneoplastic syndromes, subacute combined degeneration of cord, subacute myelo-optic neuropathy, adrenomyeloneuropathy, spinocerebellar syndromes, hereditary spastic paraparesis/primary lateral sclerosis, strokes, tumors, arteriovenous malformations, arachnoid cysts, Amold-Chiari malformations, and cervical spondylosis. Consequently, the diagnosis of MS must be made by a process that demonstrates findings consistent with MS, and also rules out other causes.

Generally, the diagnosis of MS relies on two criteria. First, there must have been two attacks at least one month apart. An attack, also known as an exacerbation, flare, or relapse, is a sudden appearance of or worsening of an MS symptom or symptoms which lasts at least 24 hours. Second, there must be more than one area of damage to central nervous system myelin sheath. Damage to sheath must have occurred at more than one point in time and not have been caused by any other disease that can cause demyelination or similar neurologic symptoms. MRI (magnetic resonance imaging) currently is the preferred method of imaging the brain to detect the presence of plaques or scarring caused by MS.

The diagnosis of MS cannot be made, however, solely on the basis of MRI. Other diseases can cause comparable lesions in the brain that resemble those caused by MS. Furthermore, the appearance of brain lesions by MRI can be quite heterogeneous in different patients, even resembling brain or spinal cord tumors in some. In addition, a normal MRI scan does not rule out a diagnosis of MS, as a small number of patients with confirmed MS do not show any lesions in the brain on MRI. These individuals often have spinal cord lesions or lesions which cannot be detected by MRI. As a result, it is critical that a thorough clinical exam also include a patient history and functional testing. This should cover mental, emotional, and language functions, movement and coordination, vision, balance, and the functions of the five senses. Sex, birthplace, family history, and age of the person when symptoms first began are also important considerations. Other tests, including evoked potentials (electrical diagnostic studies that may reveal delays in central nervous system conduction times), cerebrospinal fluid (seeking the presence of clonally-expanded immunoglobulin genes, referred to as oligoclonal bands), and blood (to rule out other causes), may be required in certain cases.

D. Combination Therapy

Combination therapies for the immune disorders listed above is also contemplated. Such therapies would include standard therapies such as anti-inflammatories and immunosuppressive agents, used in conjunction with the therapeutic methods of the present invention. Such standard therapies would be capable of negatively affecting an immune cell causing disease in a subject or to alleviate the symptoms of such disease. This process may involve contacting the cells or subject with the both agent(s) at the same time. This may be achieved with a single composition or pharmacological formulation that includes both agents, or with two distinct compositions or formulations at the same time. Alternatively, the TcR receptor therapy may precede or follow the other agent treatment by intervals ranging from minutes to weeks.

Various combinations may be employed; for example, the TcR (with or without a conjugated therapeutic agent) is “A” and the secondary immune disease therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic agents of the present invention to a patient will follow general protocols for the administration of that particular secondary therapy, taking into account the toxicity, if any, of the TcR treatment. It is expected that the treatment cycles would be repeated as necessary. It also is contemplated that various standard therapies, as well as surgical intervention, may be applied in combination with the described therapies.

### VII. Expression Systems

Prokaryote- and/or eukaryote-based systems can be used to produce nucleic acid sequences, or their cognate polypeptides, proteins and peptides. The present invention contemplates the use of such an expression system to produce the TcR that bind PR1.

One powerful expression technology employs the insecT-cell/baculovirus system. The insecT-cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition, numerous other expression systems exists which are commercially and widely available. One example of such a system is the STRATAGENE®'S COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

A. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention (Muzyczka, 1992). AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors in vaccines due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding an antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990). Lentiviruses also have been explored as vaccine vectors (VandenDriessche et al., Blood 100(3) 813-822, 2002).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Nucleic Acid Delivery

Suitable non-viral methods for nucleic acid delivery to effect expression of compositions of the present invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Background

The HLA-A2-restricted PR1 peptide, a nonomer (VLQELNVTV), is derived from the proteinase 3 and neutrophil elastase proteins. These proteins are aberrantly expressed in myeloid leukemia cells compared to normal hematopoietic progenitor cells. Cytotoxic T lymphocytes (CTL) that are specific for PR1 kill AML, CML and MDS cells but not normal bone marrow cells. In a recent phase I/II vaccine study, the PR1 peptide has been administered to patients with CML, AML and MDS and PR1-specific CTL immunity has been elicited in 47% of patients, and clinical responses have been observed in 26%. Therefore, the PR1 peptide is a leukemia-associated antigen with promising therapeutic potential in the treatment of patients with various forms of myeloid leukemia.

The inventor now reports on the sequence of two T cell receptors that bind specifically to the PR1/HLA-A2 ligand. The T cell receptor (TCR) on CD8+ cytotoxic T lymphocytes (CTL) is a disulfide-linked heterdimer composed of unique alpha and beta constituent chains that belong to the immunoglobulin (Ig) supergene family. All CTL precursors, as they mature in the thymus, express a unique TCR-αβ pair, which assembles during thymic development from germ line α and β genes, which are rearranged to form a mature receptor that is expressed on the plasma membrane. The TCR's ligand, a peptide/MHC-I complex, is created and expressed on the plasma membrane of the target cell or antigen presenting cell (APC). When bound to cognate peptide/MHC-I on the target cell, TCR on the CTL transduce a signal to the nucleus and that causes the CTL to become activated and to subsequently proliferate and kill the target cell that expresses the peptide/MHC-I ligand. TCR chains are organized much like Ig chains. Their N-terminal portions are variable and their C-terminal portions are constant. The variable (V) region of the TCR-β chain is encoded by a gene made of 3 distinct genetic elements (Vβ, D, and Jb) that are separated in the germline configuration. The TCR-α chain follows similarly, but it does not use a D gene. In the V-region domains of each chain, there are 3 complementarity-determining regions (CDRs, numbered 1 to 3), and the six combined CDRs from the two variable domains (α and β) form the antigen-binding surface of the TCR. The CDR3 region encodes the portion of the TCR chains that binds to cognate peptide in the groove of MHC-I chains, and between the CDR3 loops of the Vα and Vβ, there is a pocket that accommodates side chains from the peptide bound to the MHC-I.

### Example 2

### Methods and Results

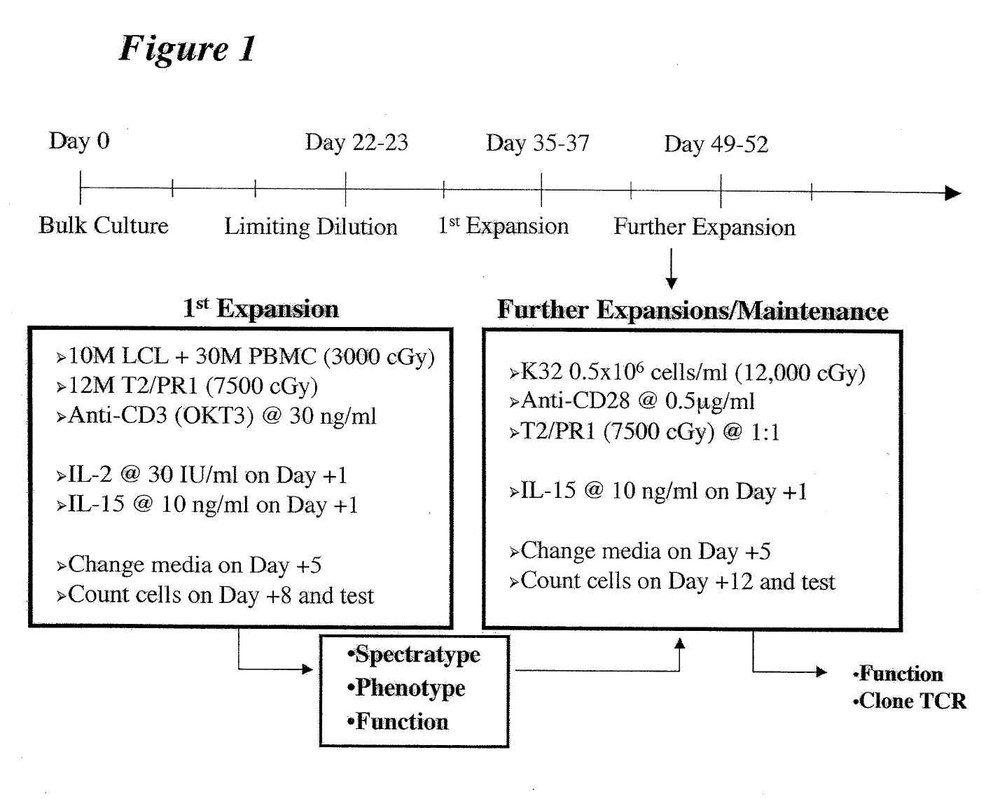

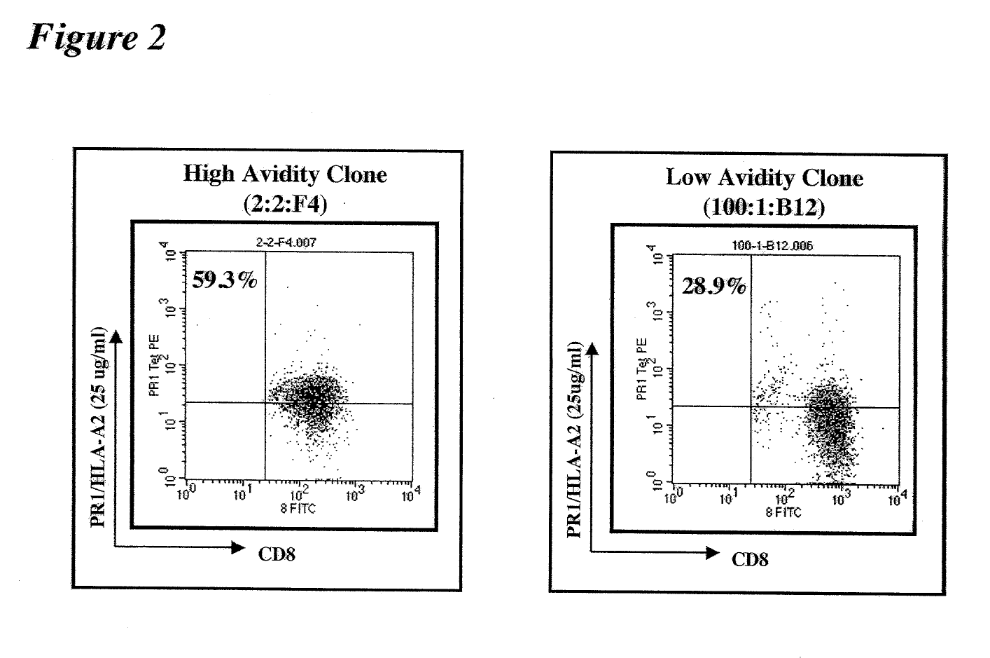

Briefly, from PBMC that were derived from a healthy HLA-A2+ donor, the inventor elicited bulk PR1-CTL cell lines after 4 to 6 weeks of weekly stimulations with the PR1 peptide+low dose IL-2 and IL-7 in vitro, as outlined in FIG. 1. These CTL were subsequently placed into limiting dilution culture and after an additional 14 to 20 days. The cells were expanded with anti-CD3/anti-CD28 beads+IL-2+feeder cells+IL-15 to sufficient numbers and were then tested. The PR1-CTLs were stained with PR1-CTL tetramer to confirm specificity (FIG. 2). Unique cDNA sequences from the CDR3-α and CDR3-β regions from two different PR1-specific CTL clones that were derived from an HLA-A2+ healthy individual were obtained. The inventor reported that one PR1-CTL clone (F4) bears a relatively high avidity TCR for the PR1/HLA-A2 ligand, while the other CTL clone (B12) bears a relatively low avidity TCR. These conclusions are supported with PR1/HLA-A2 tetramer binding data and with cytotoxicity assays that show F4 to have a higher tetramer staining fluorescence intensity and a lower threshold of activation than the B12 clone, respectively. TCR-αβ CDR3 nucleic acid sequence data is provided showing clonality at the molecular level.

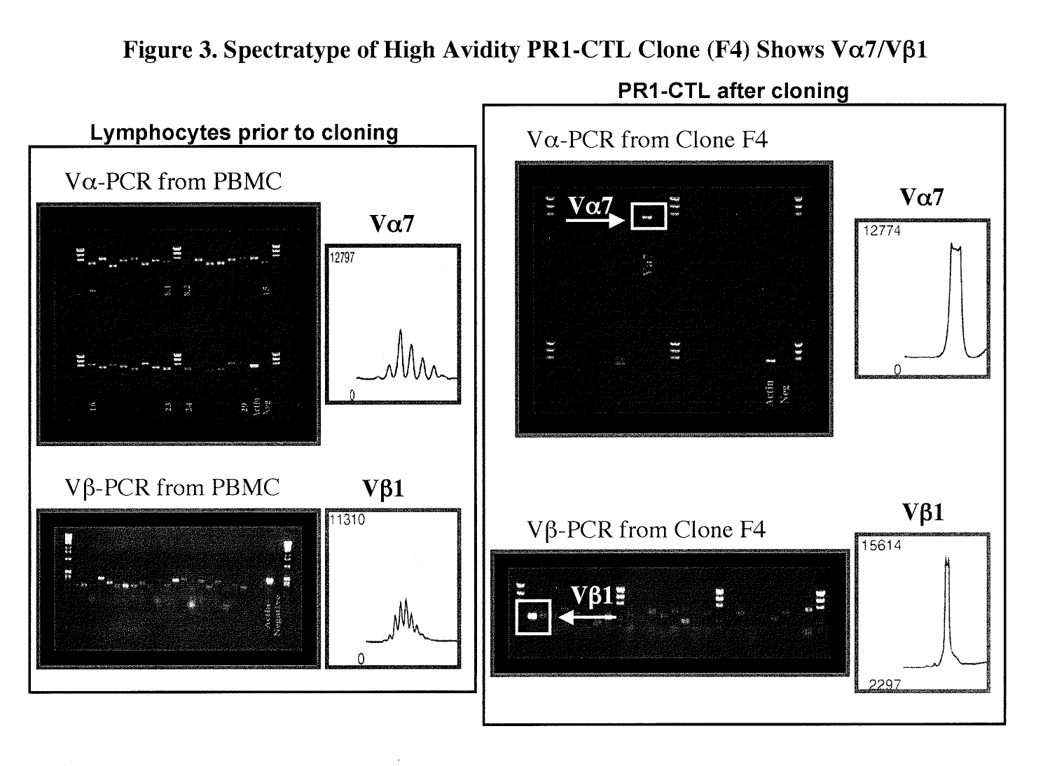

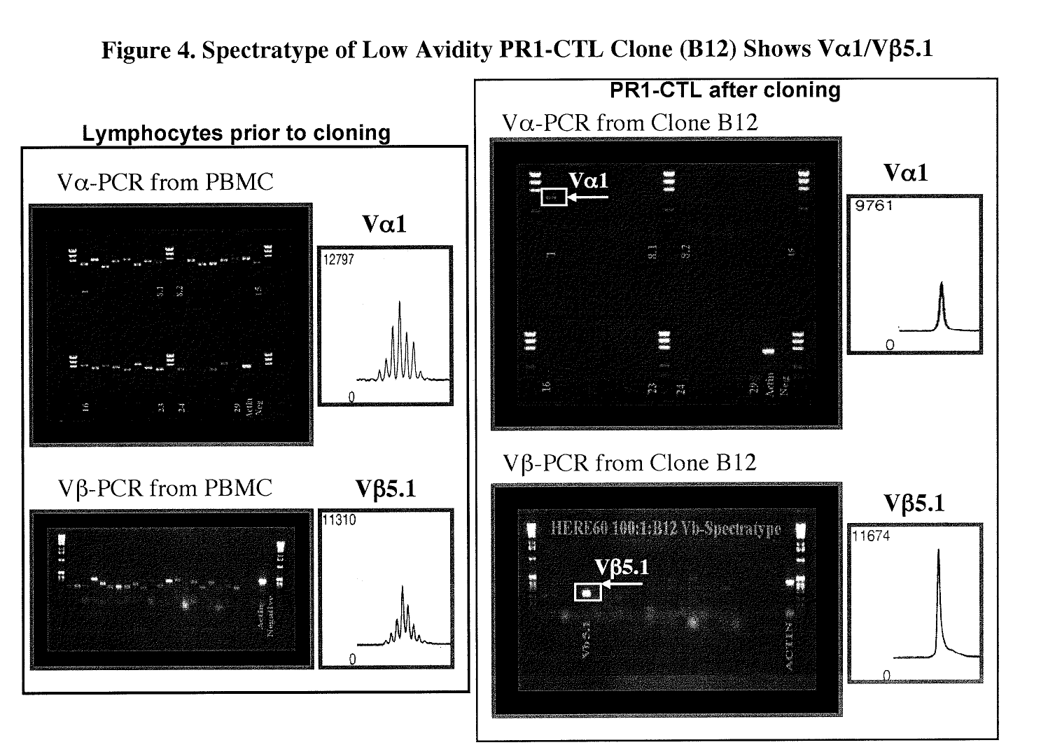

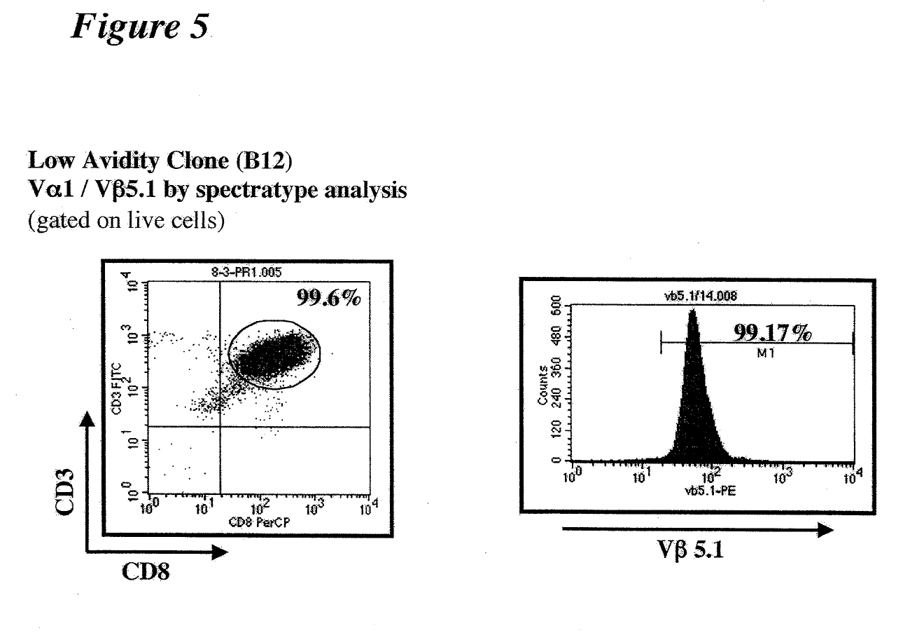

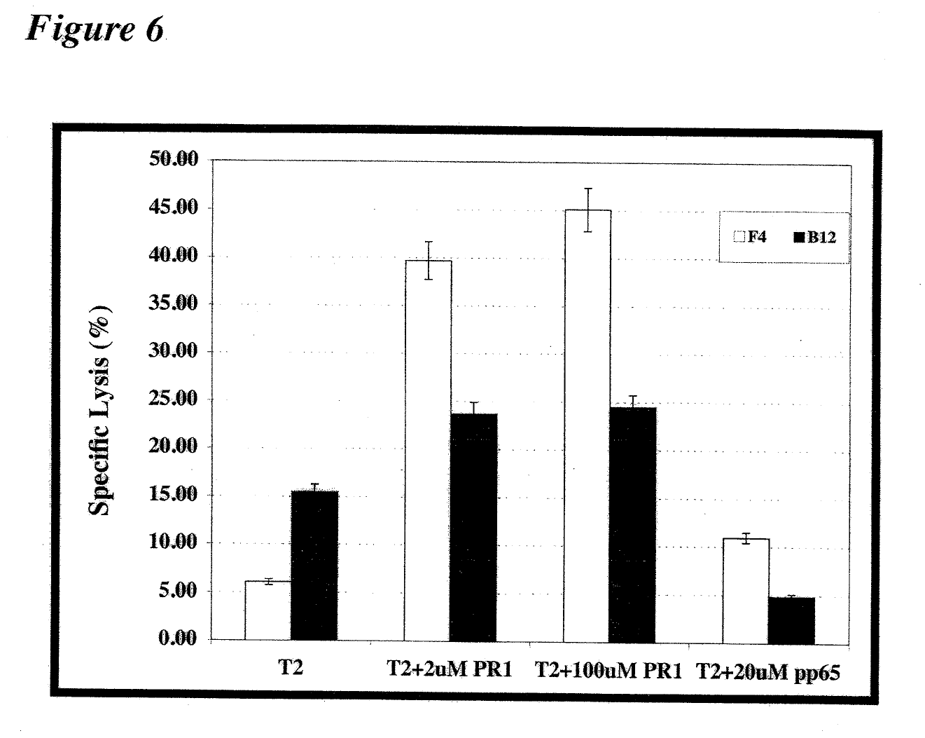

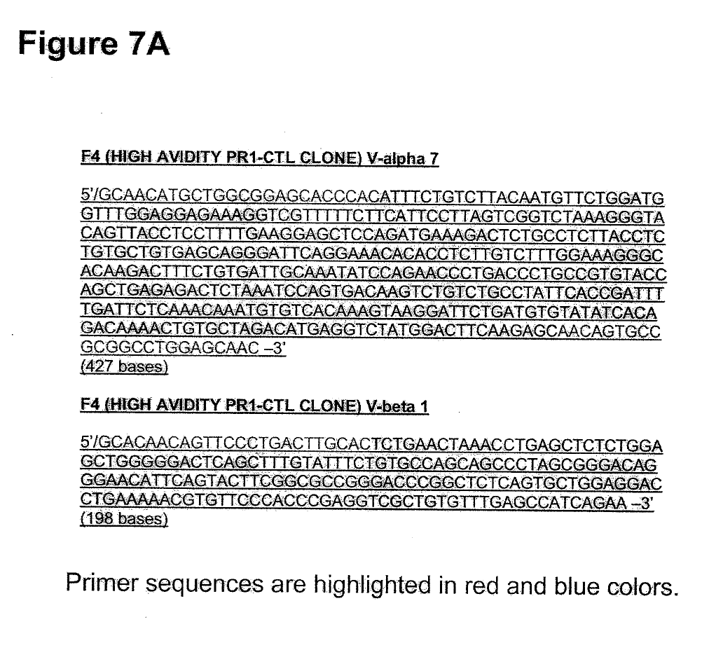

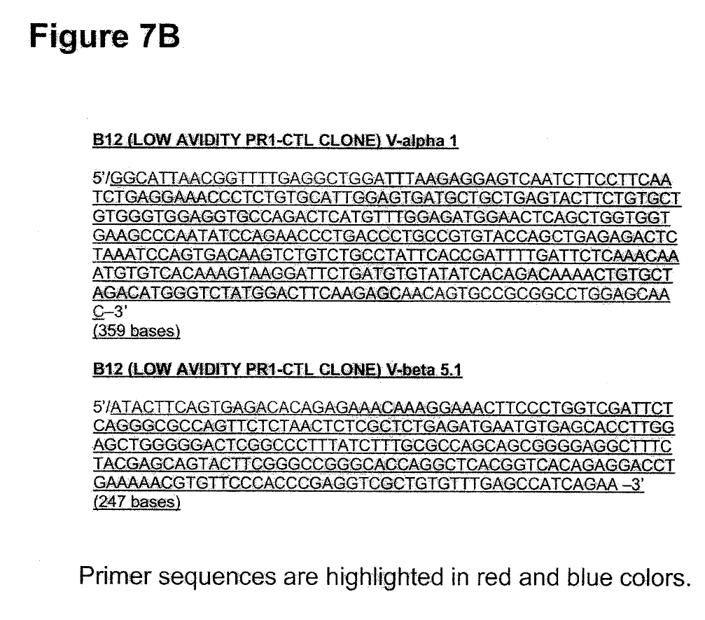

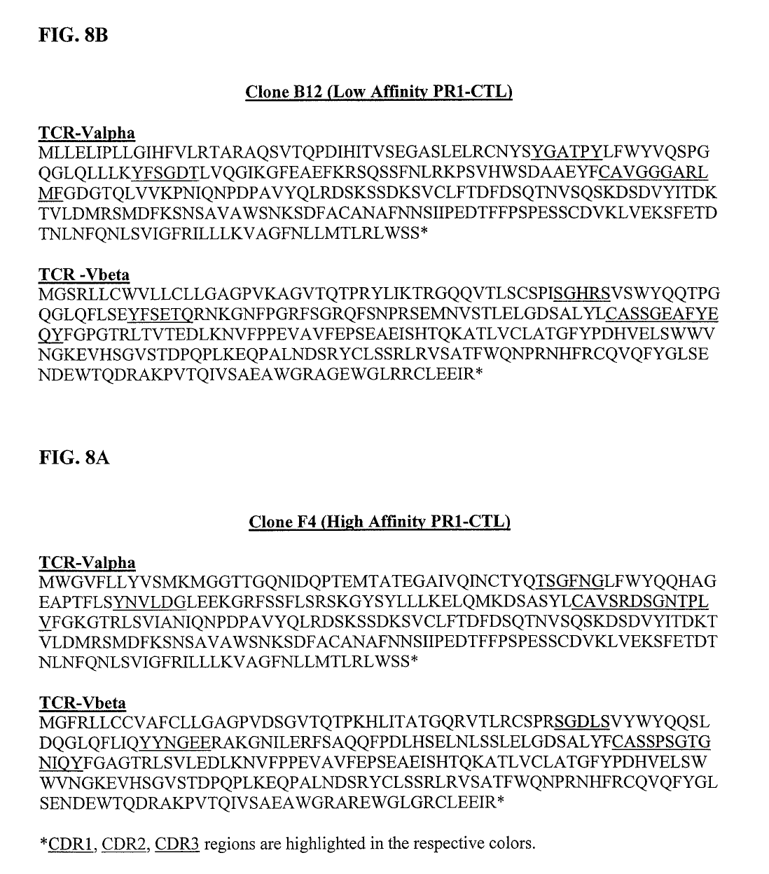

The F4 PR1-CTL shows better cytotoxicity against PR1-pulsed target cells at a fixed E:T ratio compared to the B12 clone, confirming lower activation threshold of F4 compared to B12 (FIGS. 3 and 4). Total RNA from each clone was reverse transcribed to cDNA and then forward 5′ TCR-Vβ and TCR-Vα primers from each of the V families, along with the reverse 3′ TCR-Cβ and TCR-Cα primers, respectively, were used to amplify the unique CDR3 regions from each of the clones. The CDR3 fragments were then analyzed on standard agarose gels and the amplified CDR3 products were also subjected to fragment length analysis, with both TCR-Vβ and TCR-Vα spectratype analyses to confirm that each CTL cell line was indeed clonal (FIGS. 5 and 6). To identify the sequence, the CDR3 cDNA fragments were then T/A cloned into a pcDNA3.1 vector and several colonies with inserts were sequenced in the M. D. Anderson DNA Core Facility. Single sequences were confirmed in both the forward and reverse directions. Because the inventor had determined which TCR-α and TCR-β families were used by the high and low avidity CTL clones, he could use published non-variable genomic sequences at both the 5′ and 3′ regions to design primers for the amplification of full length cDNA from each of the clones. Using primers that included the known start and stop regions for each subunit, the full length TCR-α and TCR-β was amplified by RT-PCR using RNA extracted from the high and low avidity CTL clones, and the amplified products were sequenced (FIGS. 7A and 7B). Each of these cDNA sequences was then translated to the appropriate amino acid (FIGS. 8A and 8B).

### Example 3

### CD8 T Cells with High Avidity TCR-AB Specific for Serine Protease Self-Determinants for Use in Adoptive Immunotherapy of Human Leukemia

The serine protease-derived PR1 peptide has been correlated with clinical response (CR) to interferon-α, allogeneic bone marrow transplantation or PR1-peptide vaccination in leukemia patients. However, some patients failed to demonstrate a CR, despite an immunological response. Patients with CR have functional PR1-CTL with a preferentially higher avidity T cell receptor (HTCR). These HTCR PR1-CTL were more susceptible to apoptosis by high PR1 peptide expression on leukemia cells compared to low avidity (LTCR) PR1-CTL, suggesting deletional tolerance mediated by the leukemia cells. The inventor hypothesized that the adoptive transfer of HTCR PR1-CTL could induce remission in leukemia patients. Clonal, HTCR and LTCR PR1-CTL were elicited from an HLA-A2 healthy donor using low (2 μM) or high (200 μM) concentration of PR1 peptide, respectively. TCR affinity of these clones correlated with tetramer fluorescence intensity and importantly, effector function. Jurkat and primary T cells transduced with HTCR or LTCR by lentivirus vectors expressed cell surface PR1-specific TCR complex and specifically recognized the HLA-A2/PR1 tetramers. Thus, HTCR-αβ gene transduction should have benefit for adoptive immunotherapy in leukemia patients.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. cl IX. References

The following references, to the extent that they provide exemplary procedural or other details supplementary to those set forth herein, are specifically incorporated herein by reference.
U.S. Pat. No. 4,196,265
U.S. Pat. No. 4,435,386
U.S. Pat. No. 4,436,727
U.S. Pat. No. 4,436,728
U.S. Pat. No. 4,505,899
U.S. Pat. No. 4,505,900
U.S. Pat. No. 4,520,019
U.S. Pat. No. 4,554,101
U.S. Pat. No. 4,579,945
U.S. Pat. No. 4,684,611
U.S. Pat. No. 4,797,368
U.S. Pat. No. 4,866,034
U.S. Pat. No. 4,877,611
U.S. Pat. No. 4,879,236
U.S. Pat. No. 4,950,645
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,609,870
U.S. Pat. No. 5,610,042
U.S. Pat. No. 5,641,515
U.S. Pat. No. 5,656,610
U.S. Pat. No. 5,693,762
U.S. Pat. No. 5,702,932
U.S. Pat. No. 5,736,524
U.S. Pat. No. 5,739,169
U.S. Pat. No. 5,780,448
U.S. Pat. No. 5,785,970
U.S. Pat. No. 5,789,215
U.S. Pat. No. 5,801,005
U.S. Pat. No. 5,824,311
U.S. Pat. No. 5,830,880
U.S. Pat. No. 5,846,225
U.S. Pat. No. 5,846,233
U.S. Pat. No. 5,846,945
U.S. Pat. No. 5,861,155
U.S. Pat. No. 5,871,986
U.S. Pat. No. 5,945,100
U.S. Pat. No. 5,980,912
U.S. Pat. No. 5,981,274
U.S. Pat. No. 5,994,136
U.S. Pat. No. 5,994,624
U.S. Pat. No. 6,013,516
U.S. Pat. No. 6,020,192
U.S. Pat. No. 6,027,727
U.S. Pat. No. 6,054,297
Aichele et al., J. Exp. Med., 182:261-266, 1995.
Alam et al., Nature, 381:616-620, 1996.
Alexander-Miller et al., J. Exp. Med., 184:485-492, 1996.
Alexander-Miller et al., J. Exp. Med., 188:1391-1399, 1998.
Alexander-Miller et al., Proc. Natl. Acad. Sci. USA, 93:4102-4107, 1996.
Altman et al., Science, 274:94-96, 1996.
Anderton et al., J. Exp. Med., 193:1-11, 2001.
Antin, Blood, 82:2273-2277, 1993.
Arap et al., Cancer Res., 55(6):1351-1354, 1995.
Austin-Ward and Villaseca, Rev. Med. Chil., 126(7):838-45, 1998.
Azuma et al., Cell Immunol., 116(1):123-134, 1988.
Baichwal and Sugden, In: Gene Transfer, Kucherlapati (Ed.), NY, Plenum Press, 117-148, 1986.
Bajorin et al., J. Clin. Oncol., 6(5):786-92, 1988.
Bakhshi et al., Cell, 41(3):899-906, 1985.
Ballieux et al., Clin. Exp. Immunol., 100:186-193, 1995.
Barrett et al., Blood, 95:3323-3327, 2000.
Barrett et al., Bone Marrow Transplant., 21:543-551, 1998.
Behre et al., Methods, 17:231-237, 1999.
Benjamini et al., Adv. Exp. Med. Biol., 303:71-77, 1991.
Bernhard et al., Cancer Res., 55:1099-1104, 1995.
Blomer et al., J. Virol., 71(9):6641-6649, 1997.
Bocchia et al., Blood, 85:2680-2684, 1995.
Bocchia et al., Blood, 87:3587-3592, 1996.
Boon et al., Immunol Today, 18:267-268, 1997.
Boon et al., Important Adv. Oncol., 53-69, 1994.
Bories et al., Cell, 59:959, 1989.
Borregaard and Cowland, Blood, 89:3503-3521, 1997.
Borrello et al., Blood, 95:3011-3019, 2000.
Bourdette et al., J. Immunol., 152:2510-2519, 1994.
Boussiotis et al., J. Exp. Med., 184:365-376, 1996.
Boussiotis et al., Res. Immunol., 146:140-149, 1995.
Braunschweig et al., Blood, 96:3291, 2000.
Brouwer et al., Clin. Exp. Immunol., 98:448-453, 1994.
Brutlag et al., Comput. Appl. Biosci., 6(3):237-245, 1990.
Bukowski et al., Clin. Cancer Res., 4(10):2337-47, 1998.
Burchert et al., Blood, 02:659, 2002.
Caldas et al., Nat. Genet., 8(1):27-32, 1994.
Campanelli et al., J. Exp. Med., 172:1709-1715, 1990.
Capaldi et al., Biochem. Biophys. Res. Comm., 74(2):425-433, 1977.
Celis et al., Proc. Natl. Acad. Sci. USA, 91:2105-2109, 1994.
Chen and Okayama, Mol. Cell Biol., 7(8):2745-2752, 1987.
Chen et al., Biochem. Biophys. Res. Commun., 200:1130-1135, 1994.
Cheng et al., Cancer Res., 54(21):5547-5551, 1994a.
Chou and Fasman, Adv. Enzymol., 47:45-148, 1978b.
Chou and Fasman, Annu. Rev. Biochem., 47:251-276, 1978a.
Chou and Fasman, Biochemistry, 13(2):211-222, 1974b.
Chou and Fasman, Biophys. J., 26(3):385-399, 1979.
Christodoulides et al., Microbiology, 144(Pt 11):3027-37, 1998.
Clark et al., Blood, 98:2887-2893, 2001.
Cleary and Sklar, Proc. Natl. Acad. Sci. USA, (21):7439-7443, 1985.
Cleary et al., J. Exp. Med., 164(1):315-320, 1986.
Collins, Jr. et al., J. Clin. Oncol., 15:433-444, 1997.
Cortes et al., Leukemia, 12:455-462, 1998.
Cotten et al., Proc. Natl. Acad. Sci. USA, 89(13):6094-6098, 1992.
Coupar et al., Gene, 68:1-10, 1988.
Culver et al., Science, 256(5063):1550-1552, 1992.
Curiel, Nat. Immun., 13(2-3):141-164, 1994.
Davidson et al., J. Immunother., 21(5):389-98, 1998.
den Haan et al., Science, 268:1476-1480, 1995.
den Haan et al., Science, 279:1054-1057, 1998.
Dengler et al., Br. J. Haematol., 89:250-257, 1995.
Dermime et al., Blood, 86, 1995.
Dermime et al., Bone Marrow Transplant., 19:989-999, 1997.
Devergie et al., Bone Marrow Transplant., 20:11-9, 1997.
Dickinson et al., Nat. Med., 8:410-414, 2002.
Dillman, Cancer Biother. Radiopharm., 14(1):5-10, 1999.
Dolstra et al., J. Immunol., 158:560-565, 1997.
Drobyski et al., Blood, 83:1980-1987, 1994.
Dudley et al., Science, 298:850-854, 2002.
Dunbar et al., Curr. Biol., 8:413-416, 1998.
Faber et al., Biol. Blood Marrow Transplant., 2:31-36, 1996.
Faber et al., Blood, 86:2821-2828, 1995.
Faber et al., J. Clin. Invest., 96:877-883, 1995.
Faderl et al., Ann. Intern. Med., 131:207-219, 1999.
Fechheimer et al., Proc. Natl. Acad. Sci. USA, 84:8463-8467, 1987.
Fetrow and Bryant, Biotechnology, 11(4):479-484, 1993.
Fraley et al., Proc. Natl. Acad. Sci. USA, 76:3348-3352, 1979.
Franssen et al., Kidney Int., 57:2195-1206, 2000.
Franssen et al., Lancet., 347:116, 1996.
Friedmann, Science, 244:1275-1281, 1989.
Gale et al., Ann. Intern. Med., 120:646-652, 1994.
Gallimore et al., J. Exp. Med., 187:1383-1393, 1998.
Gao et al., Blood, 95:2198-2203, 2000.
Garlie et al., J. Immunother., 22:336-345, 1999.
Gaschet et al., J. Clin. Invest., 98:100-107, 1996.
Gaugler et al., J. Exp. Med., 179:921-930, 1994.
Giralt and Kolb, Curr. Opin. Oncol., 8:96-102, 1996.
Gopal, Mol. Cell Biol., 5:1188-1190, 1985.
Goulmy et al., N. Engl. J. Med., 334:281-285, 1996.
Graham and Van Der Eb, Virology, 52:456-467, 1973.
Grossman and Paul, Proc. Natl. Acad. Sci. USA, 89:10365-10369, 1992.
Grunhaus and Horwitz, Seminar in Virology, 3:237-252, 1992.
Hanibuchi et al., Int. J. Cancer, 78(4):480-485, 1998.
Harlan and Weintraub, J. Cell Biol., 101:1094-1099, 1985.
Harlow and Lane, Antibodies: A Laboratory Manual. Cold Spring Harbor Laboratory Press, Cold Spring harbor, NY, 553-612, 1988.
Hellstrand et al., Acta Oncol., 37(4):347-353, 1998.
Hensel et al., Bone Marrow Transplant., 23:71-78, 1999.
Hermonat and Muzycska, Proc. Natl. Acad. Sci. USA, 81:6466-6470, 1984.
Herr et al., J. Immunol. Methods, 191:131-142, 1996.
Herr et al., J. Immunol. Methods, 203:141-152, 1997.
Horowitz et al., Bone Marrow Transplant., 17(Suppl 3):S5-6, 1996.
Horwich et al. J. Virol., 64:642-650, 1990.
Hui and Hashimoto, Infect. Immun., 66(11):5329-36, 1998.
Husson et al., J. Bacteriol., 172(2):519-524, 1990.
Hussussian et al., Nat. Genet., 8(1):15-21, 1994.
Irie and Morton, Proc. Natl. Acad. Sci. USA, 83(22):8694-8698, 1986.
Irie et al., Lancet., 1(8641):786-787, 1989.
Jacobs et al., Nature, 327(6122):532-535, 1987.
Jameson and Wolf, Comput. Appl. Biosci., 4(1):181-186, 1988.
Janetzki et al., J. Immunol. Methods, 234:1-12, 2000.
Jennette et al., Semin. Diagn. Pathol., 18:3-13, 2001.
Johnson et al., In: Biotechnology And Pharmacy, Pezzuto et al. (Eds.), Chapman and Hall, NY, 1993.
Ju et al., J. Neuropathol. Exp. Neurol., 59(3):241-50, 2000.
Kaeppler et al., PlanT-cell Reports, 9:415-418, 1990.
Kamb et al., Nat. Genet., 8(1):23-2, 1994.
Kamb et al., Science, 2674:436-440, 1994.
Kaneda et al., Science, 243:375-378, 1989.
Kato et al, J. Biol. Chem., 266:3361-3364, 1991.
Kelleher and Vos, Biotechniques, 17(6): 1110-7, 1994.
Kerr et al., Br. J. Cancer, 26(4):239-257, 1972.
Klinman, Curr. Prot. Immunol., 6.19:18, 1994.
Kochenderfer and Molldrem, Curr. Oncol. Rep. 3:193-200, 2001.
Kochenderfer et al., Blood, 100:3639-3645, 2002.
Kolb and Holler, Curr. Opin. Oncol., 9:139-145, 1997.
Kolb et al., Blood, 86:2041-2050, 1995.
Kolb et al., Bone Marrow Transplant, 17:449-452, 1996.
Kwak et al., Proc. Natl. Acad. Sci. USA, 93:10972-10977, 1996.
Kyte and Doolittle, J. Mol. Biol., 57(1):105-32, 1982.
Lalvani et al., J. Exp. Med., 186:859-865, 1997.
Laughlin et al., J. Virol., 60(2):515-524, 1986.
Lebkowski et al., Mol. Cell. Biol., 8(10):3988-3996, 1988.
Lee et al., Nat. Med., 5:677-685, 1999.
Lehmann et al., Eur. J. Immunol., 25:340-347, 1995.
Levine et al., J. Hematother., 7:437-448, 1998.
Lewin et al., Blood, 100:2235-2242, 2002.
Lotte et al., Adv. Tuberc. Res., 21:107-93; 194-245, 1984.
Lubbert et al., Leukemia, 13:1420-1427, 1999.
Luelmo, Am. Rev. Respir. Dis., 125(3 Pt 2):70-72, 1982.
Mackinnon et al., Bone Marrow Transplant., 15:591-594, 1995.
Maeurer et al., J. Clin. Invest., 98:1633-1641, 1996.
Mann et al., Cell, 33:153-159, 1983.
Marchand et al., Int. J. Cancer, 63:883-885, 1995.
Marijt et al., Blood 82:3778-3785, 1995.
Marincola et al., J. Immunother. Emphasis Tumor Immunol., 19:192-205, 1996.
Martin et al., Nature, 345(6277):739-743, 1990.
Matulonis et al., Blood, 85:2507-2515, 1995.
Mavroudis et al., Bone Marrow Transplant., 17:793-799, 1996.
Mayet et al., Eur. J. Clin. Invest., 27:893-899, 1997.
McLaughlin et al., J. Virol., 62(6):1963-1973, 1988.
Miller et al., Am. J. Clin. Oncol., 15(3):216-221, 1992.
Mitchell et al., Ann. NY Acad. Sci., 690:153-166, 1993.
Mitchell et al., J. Clin. Oncol., 8(5):856-869, 1990.
Molldrem et al., Blood, 88:2450-2457, 1996.
Molldrem et al., Blood, 90:1858, 1998.
Molldrem et al., Blood, 90:2529-2534, 1997.
Molldrem et al., Br. J. Haematol., 102:1314-1322, 1998.
Molldrem et al., Cancer Res., 59:2675-2681, 1999.
Molldrem et al., Curr. Opin. Hematol., 9:503-508, 2002.
Molldrem et al., J. Clin. Invest., 111:639-647, 2003.
Molldrem et al., Nat. Med., 6:1018-1023, 2000.
Molldrem et al., Oncogene, 21:8668-8673, 2002.
Morgan et al., J. Immunol., 160:643-651, 1998.
Mori et al., Cancer Res., 54(13):3396-3397, 1994.
Morton et al., Arch. Surg., 127:392-399, 1992.
Muller-Berat et al., Clin. Immunol. Immunopathol., 70:51-59, 1994.
Muzyczka, Curr. Topics Microbiol. Immunol., 158:97-129, 1992.
Nakamoto et al., J. Immunol., 158:5692-5697, 1997.
Naldini et al., Science, 272(5259):263-267, 1996.
Nanda and Sercarz, Cell, 82:13-17, 1995.
Nelson et al., Blood, 88:580-589, 1996.
Nestle et al., Nature Med., 4:328-332, 1998.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Okamoto et al., Proc. Natl. Acad. Sci. USA, 1(23):11045-11049, 1994.
Omirulleh et al., Plant Mol. Biol., 21(3):415-28, 1993.
Orlow et al., Cancer Res, 54(11):2848-2851, 1994.
Papadopoulos et al., Blood, 90:4938-4946, 1997.
Pardoll, Current Opin. Immunol., 14:619-623, 1992.
Pardoll, Nature Rev. Immunol., 2:227-238, 2002.
Pardoll, Nature, 369:357, 1994.
Parker et al., J. Immunol., 152:163, 1994.
Parkhurst et al., J. Immunol., 157:2539-2548, 1996.
Paskind et al., Virology, 67:242-248, 1975.
PCT Appln, WO 91/16347
PCT Appln. WO 94/09699
PCT Appln. WO 95/06128
Pietras et al., Oncogene, 17(17):2235-49, 1998.
Pinilla-Ibarz et al., Blood, 95:1781-1787, 2000.
Potrykus et al., Mol. Gen. Genet., 199:183-188, 1985.
Qin et al., Proc. Natl. Acad. Sci. USA, 95(24):14411-14416, 1998.
Rabinovich et al., Science, 265(5177):1401-1404, 1994.
Rammensee et al., Immunogenetics, 41:178-228, 1995.
Ravindranath and Morton, Intern. Rev. Immunol., 7: 303-329, 1991.
Rees et al., Proc. Natl. Acad. Sci. USA, 96:9781-9786, 1999.
Reich-Zeliger et al., Immunity, 13:507-515, 2000.
Remington's Pharmaceutical Sciences, 15^{th} ed., pages 1035-1038 and 1570-1580, Mack Publishing Company, Easton, Pa., 1980.
Restifo et al., J. Exp. Med., 177:265-272, 1993.
Riddell and Greenberg, Annu. Rev. Immunol., 13:545-586, 1995.
Riddell and Greenberg, Cancer Treat Res., 76:337-369, 1995.
Riddell and Greenberg, Curr. Top Microbiol. Immunol., 189:9-34, 1994.
Riddell et al., Science, 257:238-241, 1992.
Ridgeway, In: Vectors: A survey of molecular cloning vectors and their uses, Rodriguez and Denhardt (Eds.), Stoneham:Butterworth, 467-492, 1988.
Rippe et al., Mol. Cell. Biol., 10:689-695, 1990.
Rosenberg and White, J. Immunother. Emphasis. Tumor Immunol., 19:81-84, 1996.
Rosenberg et al., Ann. Surg. 210(4):474-548, 1989.
Rosenberg et al., Nature Med., 4:321-327, 1998.
Rosenberg et al., N. Engl. J. Med., 319:1676, 1988.
Salter and Cresswell, Embo. J., 5:943-949, 1986.
Santini et al., J. Exp. Med., 191:1777-1788, 2000.
Savage et al., Immunity, 10:485-492, 1999.
Savige et al., Am. J. Clin. Pathol., 111:507-513, 1999.
Scheibenbogen et al., Blood, 100:2132-2137, 2002.
Scheibenbogen et al., Int. J. Cancer, 71:932-936, 1997.
Serrano et al., Nature, 366:704-707, 1993.
Serrano et al., Science, 267(5195):249-252, 1995.
Sette et al., J. Immunol., 153:5586-5592, 1994.
Shlomchik et al., Science, 285:412-415, 1999.
Sibelius et al., J. Exp. Med., 187:497-503, 1998.
Snapper et al., Proc. Natl. Acad. Sci. USA, 85(18):6987-6991, 1988.
Stark, G. R., Kerr, I. M., Williams, B. R., Silverman, R. H. & Schreiber, R. D. How cells respond to interferons. Annu Rev Biochem 67, 227-64 (1998).
Sturrock et al., J. Biol. Chem., 267:21193, 1992.
Takada et al., J. Clin. Microbiol., 33(3):658-660, 1995.
Tang et al., J. Biol. Chem. 271(26):15682-15686, 1996.
Tanaka et al., Cancer Res., 57:4465-4468, 1997.
Tanaka et al., Genes Cells, 3:29-37, 1998.
Temin, In: Gene Transfer, Kucherlapati (Ed.), NY, Plenum Press, 149-188, 1986.
Teshima et al., Nat. Med., 8:575-581, 2002.
Toes et al., Int. J. Cancer, 66:686-691, 1996.
Toes et al., J. Immunol., 156:3911-3918, 1996.
Toes et al., Proc. Natl. Acad. Sci. USA, 93:7855-7860, 1996.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Tsukada et al., J. Exp. Clin. Cancer Res., 16:171-176, 1997.
van der Bruggen et al., Eur. J. Immunol., 124:3038-3043, 1994.
van der Harst et al., Blood, 83:1060-1066, 1994.
van Lochem et al., Bone Marrow Transplant., 10:181-183, 1992.
van Rhee et al., Blood, 83, 1994.
Verdijk et al., J. Immunol., 163:57-61, 1999.
Wada et al., Nucleic Acids Res. 18:2367-2411, 1990.
Wang et al., Biol. Blood Marrow Transplant., 6:118, 2000.
Weinberger et al., Science, 228:740-742, 1985.
Williams et al., J. Immunol., 152:4722-4732, 1994.
Wolf et al., Comput. Appl. Biosci., 4(1):187-191, 1988.
Wong et al., Gene, 10:87-94, 1980.
Yamamoto et al., Jpn. J. Cancer Res., 79:866-873, 1988.
Yee et al., J. Immunol., 162:2227-2234, 1999.
Yin et al., J. Biol. Resp. Modif., 8:190-205, 1989.
Zarour et al., J. Invest. Dermatol., 107:63-67, 1996.
Zeh et al., J. Immunol., 162:989-994, 1999.
Zufferey et al., Nat. Biotechnol., 15(9):871-875, 1997.

1. A single chain T-cell receptor comprising an α chain comprising a CDR3α-encoding segment comprising SEQ ID NO:1 or 5 and a β chain comprising a CDR3β-encoding segment comprising SEQ ID NO:3 or 7.

2. The T-cell receptor of claim 1, wherein said receptor is purified and isolated.

3. The T-cell receptor of claim 1, wherein said receptor lacks membrane-spanning regions.

4. The T-cell receptor of claim 1, wherein said receptor is soluble.

5. The T-cell receptor of claim 1, wherein said receptor is fused to a non-T-cell peptide or polypeptide segment.

6. The T-cell receptor of claim 1, wherein said receptor is linked to a diagnostic reagent.

7. The T-cell receptor of claim 6, wherein said diagnostic reagent is a fluorophore, a chromophore, a dye, a radioisotope, a chemilluminescent molecule, a paramagnetic ion, or a spin-trapping reagent.

8. The T-cell receptor of claim 1, wherein said receptor is linked to a therapeutic reagent.

9. The T-cell receptor of claim 8, wherein the therapeutic reagent is a cytokine, a chemotherapeutic, a radiotherapeutic, a hormone, an antibody Fc fragment, a TLR agonist, a CpG-containing molecule, or an immune co-stimulatory molecule.

10. The T-cell receptor of claim 1, further comprising a dimerization or multimerization sequence.

11. A nucleic acid encoding a single chain T-cell receptor comprising an α chain comprising a CDR3α-encoding segment comprising SEQ ID NO:1 or 5 and a β chain comprising a CDR3β-encoding segment comprising SEQ ID NO:3 or 7.

12. The nucleic acid of claim 11, wherein said CDR3α-encoding segment is encoded by SEQ ID NO:2 or 6 and said CDR3β-encoding segment is encoded by SEQ ID NO:4 or 8.

13. The nucleic acid of claim 11, further comprising a nucleic acid segment encoding a non-T-cell peptide or polypeptide.

14. The nucleic acid of claim 11, further comprising a promoter sequence positioned 5′ to the nucleic acid encoding the T-cell receptor.

15. The nucleic acid of claim 14, wherein said promoter is active in eukaryotic cells.

16. The nucleic acid of claim 14, wherein said promoter is active in prokaryotic cells.

17. The nucleic acid of claim 11, wherein said nucleic acid is located in a replicable vector.

18. The nucleic acid of claim 17, wherein said replicable vector is a non-viral vector.

19. The nucleic acid of claim 17, wherein said replicable vector is a viral vector.

20. The nucleic acid of claim 11, further comprising a linker-encoding segment, wherein said linker-encoding segment is located between said CDR3α-encoding segment and said CDR3β-encoding segment.

21. The nucleic acid of claim 20, wherein said linker-encoding segment encodes a helix-turn-helix motif.

22. The nucleic acid of claim 11, further comprising a segment encoding a dimerization or multimerization sequence.

23. An artificial T-cell receptor comprising an α chain comprising a CDR3α-encoding segment comprising SEQ ID NO:1 or 5 and a β chain comprising a CDR3β-encoding segment comprising SEQ ID NO:3 or 7, wherein said α and β chains are linked by a synthetic linker.

24-39. (canceled)

40. A method of making a recombinant T-cell receptor comprising (a) introducing into a host cell (i) a T-cell α chain comprising a CDR3α-encoding segment comprising SEQ ID NO:1 or 5 in a cell and (ii) a T-cell β chain comprising a CDR3β-encoding segment comprising SEQ ID NO:3 or 7 in a cell; and (b) culturing said host cell under conditions supporting expression of said α and β chains.

41-42. (canceled)

43. A method of detecting abnormal cells in a sample suspected of containing abnormal cells comprising contacting said sample with a single chain T-cell receptor comprising an α chain comprising a CDR3α-encoding segment comprising SEQ ID NO:1 or 5 and a β chain comprising a CDR3β-encoding segment comprising SEQ ID NO:3 or 7, or an artificial T cell receptor comprising an α chain comprising a CDR3α-encoding segment comprising SEQ ID NO:1 or 5 and a β chain comprising a CDR3β-encoding segment comprising SEQ ID NO:3 or 7, wherein said α and β chains are linked by a synthetic linker.

44-55. (canceled)

56. A method of treating a subject with cancer, myeloid dysplastic disease or autoimmune disease comprising administering to said subject a single chain T-cell receptor comprising an α chain comprising a CDR3α-encoding segment comprising SEQ ID NO:1 or 5 and a ⊕ chain comprising a CDR3β-encoding segment comprising SEQ ID NO:3 or 7, or an artificial T cell receptor comprising an α chain comprising a CDR3α-encoding segment comprising SEQ ID NO:1 or 5 and a β chain comprising a CDR3β-encoding segment comprising SEQ ID NO:3 or 7, wherein said α and β chains are linked by a synthetic linker.

57-80. (canceled)

81. A purified and isolated nucleic acid segment encoding a CDR3α comprising the amino sequence of SEQ ID NO:1, SEQ ID NO:3, SEQ ID NO:5, SEQ ID NO:7, or SEQ ID NO:5 and SEQ ID NO:7.

82. The purified and isolated nucleic acid segment of claim 81, wherein the nucleotide sequence comprises SEQ ID NO:2, SEQ ID NO:4, SEQ ID NO:6 or SEQ ID NO:8.

83-91. (canceled)
